SEQUENCE LISTING (72 sequences)

SEQ ID NO: 1 - DNA, 129 bp, Pseudomonas putida
  misc_feature: gene sequence of the bkd promoter
agtttgcgcatgagacaaaatcaccggttttttgtgtttatgcggaatgtttatctgccccgctcggcaaaggcaatcaattgagagaaaaattctcctgccggaccactaagatgtaggggacgctga

SEQ ID NO: 2 - DNA, 486 bp, Pseudomonas putida
  misc_feature: gene sequence of the BkdR regulator
ctattcgcgcaaggtcatgccattggccggcaacggcaaggctgtcttgtagcgcacctgtttcaaggcaaaactcgagcggatattcgccacacccggcaaccgggtcaggtaatcgagaaaccgctccagcgcctggatactcggcagcagtacccgcaacaggtagtccgggtcgcccgtcatcaggtagcactccatcacctcgggccgttcggcaatttcttcctcgaagcggtgcagcgactgctctacctgtttttccaggctgacatggatgaacacattcacatccagccccaacgcctcgggcgacaacaaggtcacctgctggcggatcacccccagttcttccatggcccgcacccggttgaaacagggcgtgggcgacaggttgaccgagcgtgccagctcggcgttggtgatgcgggcgttttcctgcaggctgttgagaatgccgatatcggtacgatcgagtttgcgcat

SEQ ID NO: 3 - DNA, 105 bp, Bacillus subtilis
  misc_feature: gene sequence of the ackA promoter
aacctatagtgaatgtgtctgaaaataacgacttcttattgtaagcgttatcaatacgcaagttgacttgaaaagccgacatgacaatgtttaaatggaaaagtc

SEQ ID NO: 4 - DNA, 780 bp, Bacillus subtilis
  misc_feature: gene sequence of the CodY activator
atggctttattacaaaaaacaagaattattaactccatgctgcaagctgcggcagggaaaccggtaaacttcaaggaaatggcggagacgctgcgggatgtaattgattccaatattttcgttgtaagccgcagagggaaactccttgggtattcaattaaccagcaaattgaaaatgatcgtatgaaaaaaatgcttgaggatcgtcaattccctgaagaatatacgaaaaatctgtttaatgtccctgaaacatcttctaacttggatattaatagtgaatatactgctttccctgttgagaacagagacctgtttcaagctggtttaacaacaattgtgccgatcatcggaggcggggaaagattaggaacacttattctttcgcgtttacaagatcaattcaatgacgatgacttaattctagctgaatacggcgcaacagttgtcggaatggaaatcctaagagaaaaagcagaagaaattgaagaggaagcaagaagcaaagctgtcgtacaaatggctatcagctcgctttcttacagtgagcttgaagcaattgagcacatttttgaggagcttgacggaaatgaaggtcttcttgttgcaagtaaaattgctgaccgtgtcggcattacccgttctgttattgtgaacgcactcagaaagctggagagcgccggtgttatcgagtctagatcattaggaatgaaaggtacttatatcaaggtactaaacaacaaattcctaattgaattagaaaatctaaaatctcattaa

SEQ ID NO: 5 - DNA, 106 bp, Pseudomonas putida
  misc_feature: gene sequence of the mdeA promoter
tgttgtttttatgtcagtgagcggcgcttttcgtaggcgtatttggaaaaatttaagccggtccgtggaataagcttataacaaaccacaagaggcggttgccatg

SEQ ID NO: 6 - DNA, 480 bp, Pseudomonas putida
  misc_feature: gene sequence of the MdeR regulator
tcaaatatgcttctgtgccaccggaatcacccgcttctccttcaccgccttgaacgagaagctcgaatagatctccttcacccccggcagccgctgcagtacctcgcgggtgaactcgccgaacgactccagatcccgcgccagaatctccagcaggaagtcatagcgcccggagatgttgtggcacgccacgatttcggggatatccatcagccgctgctcgaatgcccgggccatctccttgctgtgcgaatccatcatgatgctgacgaaggcggtcactccgaagcccagtgccttgggtgacaggatggcctgatagccggtgatgtagcccgactcctccagcagcttgacccgccgccagcacggcgaggtggtcagggcgacgctgtcggcgagctcggccacggtcagtcgggcattgtcttgcagcgcggccagcagtgcgcggtcggtacggtcgatggcgctaggcat

SEQ ID NO: 7 - DNA, 186 bp, Corynebacterium glutamicum
  misc_feature: gene sequence of the brnF promoter
tttttagaccttgcgcgatttcgtagcgccgataacctttatcatctggttccagggctgccttggatggcgacacctccaggcttgaatgaatctcttgcgttttttgcacactacaatcatcacacaattgccgggtagttttgttgccagtttgcgcacctcaactaggctattgtgcaatat

SEQ ID NO: 8 - DNA, 456 bp, Corynebacterium glutamicum
  misc_feature: gene sequence of the Lrp regulator
atgaagctagattccattgatcgcgcaattattgcggagcttagcgcgaatgcgcgcatctcaaatctcgcactggctgacaaggtgcatctcactccgggaccttgcttgaggagggtgcagcgtttggaagccgaaggaatcattttgggctacagcgcggacattcaccctgcggtgatgaatcgtggatttgaggtgaccgtggatgtcactctcagcaacttcgaccgctccactgtagacaattttgaaagctccgttgcgcagcatgatgaagtactggagttgcacaggctttttggttcgccagattattttgtccgcatcggcgttgctgatttggaggcgtatgagcaatttttatccagtcacattcaaaccgtgccaggaattgcaaagatctcatcacgttttgctatgaaagtggtgaaaccagctcgcccccaggtgtga

SEQ ID NO: 9 - DNA, 89 bp, Escherichia coli
  misc_feature: gene sequence of the cysP promoter
aacttattcccttttcaacttccaaatcaccaaacggtatataaaaccgttactcctttcacgtccgttataaatatgatggctattag

SEQ ID NO: 10 - DNA, 975 bp, Escherichia coli
  misc_feature: gene sequence of the CysB regulator
atgaaattacaacaacttcgctatattgttgaggtggtcaatcataacctgaatgtctcatcaacagcggaaggactttacacatcacaacccgggatcagtaaacaagtcagaatgctggaagacgagctaggcattcaaattttttcccgaagcggcaagcacctgacgcaggtaacgccagcagggcaagaaataattcgtatcgctcgcgaagtcctgtcgaaagtcgatgccataaaatcggttgccggagagcacacctggccggataaaggttcactgtatatcgccaccacgcatacccaggcacgctacgcattaccaaacgtcatcaaaggctttattgagcgttatcctcgcgtttctttgcatatgcaccagggctcgccgacacaaattgctgatgccgtctctaaaggcaatgctgatttcgctatcgccacagaagcgctgcatctgtatgaagatttagtgatgttaccgtgctaccactggaatcgggctattgtagtcactccggatcacccgctggcaggcaaaaaagccattaccattgaagaactggcgcaatatccgttggtgacatataccttcggctttaccggacgttcagaactggatactgcctttaatcgcgcagggttaacgccgcgtatcgttttcacggcaacggatgctgacgtcattaaaacttacgtccggttagggctgggggtaggggtcattgccagcatggcggtggatccggtcgccgatcccgaccttgtgcgtgttgatgctcacgatatcttcagccacagtacaaccaaaattggttttcgccgtagtactttcttgcgcagttatatgtatgatttcattcagcgttttgcaccgcatttaacgcgtgatgtcgttgatgcggctgtcgcattgcgctctaatgaagaaattgaggtcatgtttaaagatataaaactgccggaaaaataa

SEQ ID NO: 11 - DNA, 270 bp, Escherichia coli
  misc_feature: gene sequence of the cadB promoter
tttttattacataaatttaaccagagaatgtcacgcaatccattgtaaacattaaatgtttatcttttcatgatatcaacttgcgatcctgatgtgttaataaaaaacctcaagttctcacttacagaaacttttgtgttatttcacctaatctttaggattaatccttttttcgtgagtaatcttatcgccagtttggtctggtcaggaaatagttatacatcatgacccggactccaaattcaaaaatgaaattaggagaagagcatg

SEQ ID NO: 12 - DNA, 1539 bp, Escherichia coli
  misc_feature: gene sequence of the CadC regulator
ttattctgaagcaagaaatttgtcgagataaggtacaacataaggaacagaagtctggaatataccattttcaatccagtaaagggtgtttgcccctgggcgtaaattaaaggcggtgagatatgcatcagctgcttcccggttcatccccttcatttcataaaccttgccaagcaacacataatttagccaggacatttcaagatcaatgccagtatttatcgcctggtaagactcatctgttttaccttttaccagagcactgaccgcttttatttgatatataatggacaggttgttcaattccggcagtgtaacaatgttatctatttctgtgttcagtgctgctaattgtttttcatctaaaggatgttgagaatggcgcacgatatcaactaatgctttttctgctctcgcgtaggtaaattctggggatgattgaacaatctcacctaataattcactggcacggttcaatgatttatcatcgccatgcagtaaataatcatgtgcctgataaaaattagttaataacgcaccacgatgcggcaaaattttctggagcgtctcctgcattcgttgtggccacggttggtttaacgcttttgataaactctccagtaaatcattttgaatcgccagctgattaccgttagtgatgacataacgtttatccagcatggttgaaccatctgcattgtctaccaattttatcgacataaagcattgttgagcacggtattggcgctgattaacaaacgcaatagataatgttttaccggaactgctcggttcatcaatgttgtagttgattttgtcatgcaccataaaggtggagaaggtgttaagtgatgtcgccaccaaatcacccacgcctatcgcgtaagagagctgatacggggaactccagctgttacaacttttatttaccatattaatgtcaatatcgcgtggattgagcaaaatacgcgatttgctcataggaagacgtgtatcaagacttgaaaacgctaccagtgctacacagatacctaacgacaacaggaaaaaaaaccatacccaaaaggtagtgaatcgtttgcttttaactggggattgttcaggtggcgttgcggtgttttgaatgttaagactgtgggagggagaatctgtggcaggaaccgcctctggtatagggggaggcgaagatagcattatttcctctccctcttcttcgctgtaccagataaccggcaccattaatttatagccgcgctttggtacagtagcgatatagacaggactatcttcatcattatcttttaatgacttacgtagttctgagatactctgcgtcacaacgtgattggtgacaatacttctcttccagacattatcgataagttcatccctgctaagtacttcgccactgtgttgagcaaagaaaaccagaagatcgattaatctcggctcaagggtaagttgacgcccattgcggctaatttggtttatggacggagtaacaagccattcgccaacgcgaactacaggttgttgcat

SEQ ID NO: 13 - DNA, 16 bp, Corynebacterium glutamicum
  misc_feature: gene sequence of the metY promoter
tagaccaagatgttca

SEQ ID NO: 14 - DNA, 642 bp, Corynebacterium glutamicum
  misc_feature: gene sequence of the McbR regulator
ctaaattgagtagtccgcaggtggagccgacaacaactgccgagccaaatcgcgagccgtctcaagaggactgatgttgtggaccaatcgagatccagcaagtccaccatcaaggaacaccaacagctgattcgcctgggtggtgcctgggtagccgttcttctcagtgagcaaatcagtcagagtcttatgacaccactcgcggtgctctaacactgctgcaacaatgcccttttcgctatcagtttcggggcgagggtactcactagccgcattctgaaagtgcgagccgcggaaatctttttctggttcttcctcaatgcactgatcaaagaacgcgatgattttatcttccggatccttcataccgacggtgcgctcacgccacgcttcacgccacagctgatcgaggttctccaggtatgcaataaccaaggcgtccttcgatccgaaaagggaatagaggctcgccttcgccacgtcagcttcacggaggatacgatcaataccgatgacgcgaataccttctgtggtgaaaaggttggttgcgctatcgaggagacgctgtcgggggcttggtcgattgcgacgacggtttgccccggcacttgttttactcttgcctgaagcgctagcagccac

SEQ ID NO: 15 - DNA, 101 bp, Escherichia coli
  misc_feature: gene sequence of the argO promoter
cttattagtttttctgattgccaattaatattatcaatttccgctaataacaatcccgcgatatagtctctgcatcagatacttaattcggaatatccaac

SEQ ID NO: 16 - DNA, 894 bp, Escherichia coli
  misc_feature: gene sequence of the ArgP regulator
atgaaacgcccggactacagaacattacaggcactggatgcggtgatacgtgaacgaggatttgagcgcgcggcacaaaagctgtgcattacacaatcagccgtctcacagcgcattaagcaactggaaaatatgttcgggcagccgctgttggtgcgtaccgtaccgccgcgcccgacggaacaagggcaaaaactgctggcactgctgcgccaggtggagttgctggaagaagagtggctgggcgatgaacaaaccggttcgactccgctgctgctttcactggcggtcaacgccgacagtctggcgacgtggttgcttcctgcactggctcctgtgttggctgattcgcctatccgcctcaacttgcaggtagaagatgaaacccgcactcaggaacgtctgcgccgcggcgaagtggtcggcgcggtgagtattcaacatcaggcgctgccgagttgtcttgtcgataaacttggtgcgctcgactatctgttcgtcagctcaaaaccctttgccgaaaaatatttccctaacggcgtaacgcgttcggcattactgaaagcgccagtggtcgcgtttgaccatcttgacgatatgcaccaggcctttttgcagcaaaacttcgatctgcctccaggcagcgtgccctgccatatcgttaattcttcagaagcgttcgtacaacttgctcgccagggcaccacctgctgtatgatcccgcacctgcaaatcgagaaagagctggccagcggtgaactgattgacttaacgcctgggctatttcaacgacggatgctctactggcaccgctttgctcctgaaagccgcatgatgcgtaaagtcactgatgcgttactcgattatggtcacaaagtccttcgtcaggattaa

SEQ ID NO: 17 - DNA, 110 bp, Corynebacterium glutamicum
  misc_feature: gene sequence of the lysE promoter
gcaaagtgtccagttgaatggggttcatgaagctatattaaaccatgttaagaaccaatcattttacttaagtacttccataggtcacgatggtgatcatggaaatcttc

SEQ ID NO: 18 - DNA, 873 bp, Corynebacterium glutamicum
  misc_feature: gene sequence of the LysG regulator
atgaaccccattcaactggacactttgctctcaatcattgatgaaggcagcttcgaaggcgcctccttagccctttccatttccccctcggcggtgagtcagcgcgttaaagctctcgagcatcacgtgggtcgagtgttggtatcgcgcacccaaccggccaaagcaaccgaagcgggtgaagtccttgtgcaagcagcgcggaaaatggtgttgctgcaagcagaaactaaagcgcaactatctggacgccttgctgaaatcccgttaaccatcgccatcaacgcagattcgctatccacatggtttcctcccgtgttcaacgaggtagcttcttggggtggagcaacgctcacgctgcgcttggaagatgaagcgcacacattatccttgctgcggcgtggagatgttttaggagcggtaacccgtgaagctaatcccgtggcgggatgtgaagtagtagaacttggaaccatgcgccacttggccattgcaaccccctcattgcgggatgcctacatggttgatgggaaactagattgggctgcgatgcccgtcttacgcttcggtcccaaagatgtgcttcaagaccgtgacctggacgggcgcgtcgatggtcctgtggggcgcaggcgcgtatccattgtcccgtcggcggaaggttttggtgaggcaattcgccgaggccttggttggggacttcttcccgaaacccaagctgctcccatgctaaaagcaggagaagtgatcctcctcgatgagatacccattgacacaccgatgtattggcaacgatggcgcctggaatctagatctctagctagactcacagacgccgtcgttgatgcagcaatcgagggattgcggccttag

SEQ ID NO: 19 - DNA, 198 bp, Escherichia coli
  misc_feature: fadE promoter
gtaccggataccgccaaaagcgagaagtacgggcaggtgctatgaccaggactttttgacctgaagtgcggataaaaacagcaacaatgtgagctttgttgtaattatattgtaaacatattgctaaatgtttttacatccactacaaccatatcatcacaagtggtcagacctcctacaagtaaggggcttttcgtt

SEQ ID NO: 20 - DNA, 720 bp, Escherichia coli
  misc_feature: FadR regulator
atggtcattaaggcgcaaagcccggcgggtttcgcggaagagtacattattgaaagtatctggaataaccgcttccctcccgggactattttgcccgcagaacgtgaactttcagaattaattggcgtaacgcgtactacgttacgtgaagtgttacagcgtctggcacgagatggctggttgaccattcaacatggcaagccgacgaaggtgaataatttctgggaaacttccggtttaaatatccttgaaacactggcgcgactggatcacgaaagtgtgccgcagcttattgataatttgctgtcggtgcgtaccaatatttccactatttttattcgcaccgcgtttcgtcagcatcccgataaagcgcaggaagtgctggctaccgctaatgaagtggccgatcacgccgatgcctttgccgagctggattacaacatattccgcggcctggcgtttgcttccggcaacccgatttacggtctgattcttaacgggatgaaagggctgtatacgcgtattggtcgtcactatttcgccaatccggaagcgcgcagtctggcgctgggcttctaccacaaactgtcggcgttgtgcagtgaaggcgcgcacgatcaggtgtacgaaacagtgcgtcgctatgggcatgagagtggcgagatttggcaccggatgcagaaaaatctgccgggtgatttagccattcaggggcgataa

SEQ ID NO: 21 - DNA, 169 bp, Bacillus subtilis
  misc_feature: gene sequence of the fadM promoter
ttaatttgcatagtggcaattttttgccagactgaagaggtcataccagttatgacctctgtacttataacaacaacgtaaggttattgcgctatgcaaacacaaatcaaagttcgtggatatcatctcgacgtttaccagcacgtcaacaacgcccgctaccttgaat

SEQ ID NO: 22 - DNA, 648 bp, Bacillus subtilis
  misc_feature: gene sequence of the FabR regulator
atgggcgtaagagcgcaacaaaaagaaaaaacccgccgttcgctggtggaagccgcatttagccaattaagtgctgaacgcagcttcgccagcctgagtttgcgtgaagtggcgcgtgaagcgggcattgctcccacctctttttatcggcatttccgcgacgtagacgaactgggtctgaccatggttgatgagagcggtttaatgctacgccaactcatgcgccaggcgcgtcagcgtatcgccaaaggcgggagtgtgatccgcacctcggtctccacatttatggagttcatcggtaataatcctaacgccttccggttattattgcgggaacgctccggcacctccgctgcgtttcgtgccgccgttgcgcgtgaaattcagcacttcattgcggaacttgcggactatctggaactcgaaaaccatatgccgcgtgcgtttactgaagcgcaagccgaagcaatggtgacaattgtcttcagtgcgggtgccgaggcgttggacgtcggcgtcgaacaacgtcggcaattagaagagcgactggtactgcaactgcgaatgatttcgaaaggggcttattactggtatcgccgtgaacaagagaaaaccgcaattattccgggaaatgtgaaggacgagtaa

SEQ ID NO: 23 - DNA, 152 bp, Escherichia coli
  misc_feature: gene sequence of the rhaSR promoter
ccgtcatactggcctcctgatgtcgtcaacacggcgaaatagtaatcacgacgtcaggttcttaccttaaattttcgacggaaaaccacgtaaaaaacgtcgatttttcaagatacaagcgtgaattttcaggaaatggcggtgagcatcac

SEQ ID NO: 24 - DNA, 149 bp, Escherichia coli
  misc_feature: gene sequence of the rhaBAD promoter
atcaccacaattcagcaaattgtgaacatcatcacgttcatctttccctggttcccaatggcccattttcctgtagtaacgagaacgtcgcgaattcaggcgctctttagactggtcgtaatgaaattcagcaggatcacattatgacc

SEQ ID NO: 25 - DNA, 759 bp, Escherichia coli
  misc_feature: gene sequence of the RhaR regulator
gtggcgcatcagttaaaacttctcaaagatgatttttttgccagcgaccagcaggcagtcgctgtggctgaccgttatccgcaagatgtctttgctgaacatacacatgatttttgtgagctggtgattgtctggcgcggtaatggcctgcatctggttttgcagaatattatttattgcccggagcgtctgaagctgaatcttgactggcagggggcgattccgggatttaacgccagcgcagggcaaccacactggcgcttaggtagcatggggatggcgcaggcgcggcaggttatcggtcagcttgagcatgaaagtagtcagcatgtgccgtttgctaacgaaatggctgagttgctgttcgggcagttggtgatgttgctgaatcgccatcgttacaccagtgattcgttgccgccaacatccagcgaaacgttgctggataagctgattacccggctggcggctagcctgaaaagtccctttgcgctggataaattttgtgatgaggcatcgtgcagtgagcgcgttttgcgtcagcaatttcgccagcagactggaatgaccatcaatcaatatctgcgacaggtcagagtgtgtcatgcgcaatatcttctccagcatagccgcctgttaatcagtgatatttcgaccgaatgtggctttgaagatagtaactatttttcggtggtgtttacccgggaaaccgggatgacgcccagccagtggcgtcatctcaattcgcagaaagattaa

SEQ ID NO: 26 - DNA, 849 bp, Escherichia coli
  misc_feature: gene sequence of the RhaS regulator
gtggcgcatcagttaaaacttctcaaagatgatttttttgccagcgaccagcaggcagtcgctgtggctgaccgttatccgcaagatgtctttgctgaacatacacatgatttttgtgagctggtgattgtctggcgcggtaatggcctgcatgtactcaacgatcgcccttatcgcattacccgtggcgatctcttttacattcatgctgacgataaacactcctacgcttccgttaacgatctggttttgcagaatattatttattgcccggagcgtctgaagctgaatcttgactggcagggggcgattccgggatttaacgccagcgcagggcaaccacactggcgcttaggtagcatggggatggcgcaggcgcggcaggttatcggtcagcttgagcatgaaagtagtcagcatgtgccgtttgctaacgaaatggctgagttgctgttcgggcagttggtgatgttgctgaatcgccatcgttacaccagtgattcgttgccgccaacatccagcgaaacgttgctggataagctgattacccggctggcggctagcctgaaaagtccctttgcgctggataaattttgtgatgaggcatcgtgcagtgagcgcgttttgcgtcagcaatttcgccagcagactggaatgaccatcaatcaatatctgcgacaggtcagagtgtgtcatgcgcaatatcttctccagcatagccgcctgttaatcagtgatatttcgaccgaatgtggctttgaagatagtaactatttttcggtggtgtttacccgggaaaccgggatgacgcccagccagtggcgtcatctcaattcgcagaaagattaa

SEQ ID NO: 27 - DNA, 77 bp, Anabaena sp.
  misc_feature: gene sequence of the hetC promoter
tatcggaaaaaatctgtaacatgagatacacaatagcatttatatttgctttagtatctctctcttgggtgggattc

SEQ ID NO: 28 - DNA, 76 bp, Anabaena sp.
  misc_feature: gene sequence of the nrrA promoter
gtaattgtggctagagtaacaaagactacaaaaccttgggcatgggcttgttactttgaaattcatcgacgctaag

SEQ ID NO: 29 - DNA, 77 bp, Anabaena sp.
  misc_feature: gene sequence of the devB promoter
cctcgcccctcatttgtacagtctgttacctttacctgaaacagatgaatgtagaatttataaaactagcatttgat

SEQ ID NO: 30 - DNA, 672 bp, Anabaena sp.
  misc_feature: gene sequence of the NtcA regulator
atgatcgtgacacaagataaggccctagcaaatgtttttcgtcagatggcaaccggagcttttcctcctgttgtcgaaacgtttgaacgcaataaaacgatcttttttcctggcgatcctgccgaacgagtctactttcttttgaaaggggctgtgaaactttccagggtgtacgaggcaggagaagagattacagtagcactactacgggaaaatagcgtttttggtgtcctgtctttgttgacaggaaacaagtcggataggttttaccatgcggtggcatttactccagtagaattgctttctgcaccaattgaacaagtggagcaagcactgaaggaaaatcctgaattatcgatgttgatgctgcggggtctgtcttcgcggattctacaaacagagatgatgattgaaaccttagcgcaccgagatatgggttcgagattggtgagttttctgttaattctctgtcgtgattttggtgttccttgtgcagatggaatcacaattgatttaaagttatctcatcaggcgatcgccgaagcaattggctctactcgcgttactgttactaggctactaggggatttgcgggagaaaaagatgatttccatccacaaaaagaagattactgtgcataaacctgtgactctcagcagacagttcacttaa

SEQ ID NO: 31 - DNA, 909 bp, Mycobacterium sp.
  misc_feature: gene sequence of the CbbR regulator
atgaccaacgcgcgattgcgagctctggtcgaactggcggataccggttcggtgcgcgccgctgctgagcgactcgtggtcaccgaatcttcgatctcctcggctttacgcgcattgagcaacgacatcggcatcagcttggtcgaccggcatggccgcggggtgcggctgactcctgccggcctgcgttacgtcgaatacgcgcggcggatcctcggcttgcacgacgaggcgatattggctgcccgcggagaggccgacccggagaatggctcgatccggctggctgcggtcacctccgcgggggaactgctcatccccgccgcgttggcatcgttccgtgccgcgtaccccggtgtcgttctgcatctggaggtggcggcgcgcagcttggtgtggcctatgctggcccgccacgaggtcgacctcgttgtggcgggacggccgccggacgaattggtccggaaagtgtgggtgcgcgccgtcagcccgaacgcgcttgtcgtcgtgggaccacccgcggtagcgaagggattccagcccgccaccgcgacctggctgctgcgtgagaccggatccggtacccgctctacgttgacggcactgcttgacgacctcgatgtcgcgccacctcaattggtgctcggatcgcacggcgcggtggttgccgcggcggtggccgggctgggcgtgacgttggtgtcgcgtcaggctgtgcagcgcgaactggccgccggcgcactcgtcgaactgccggtgcccggtactccgataagccggccatggcatgtggtcagccagatcagtccgacgatgtcgaccgaactgctcatcaagcacctcttgtcccagcgagacctgggctggcgcgatatcaacaccacccttcggggagccgttaccgcctga

SEQ ID NO: 32 - DNA, 120 bp, Streptomyces cattleya
  misc_feature: gene sequence of the pcbAB promoter
gtgctggtcccgcaccgggcggtggacagcttccggcggcagctgaccggccgctacttcggcggcccggacacctcccgcgagggcgtgctcttcctggccaactacgtcttcgacttc

SEQ ID NO: 33 - DNA, 807 bp, Streptomyces cattleya
  misc_feature: gene sequence of the ThnU regulator
atggacgcagacgactgttgggcgcgggcgggcaccgtgcggatccgcctgctcggcccggtggagctggcctgcggcacgcggccggtgccggtgaccgggcggcgccagttgagggtggtggccgcgctcgcgctggaggccggacgggtgctctccaccgcggggctgatcgcctcgttgtgggcggacgagccgccgcgcaccgccgcccggcagctccagaccagcgtgtggatgatccgccgggcgctcgcctcggtgggcgcgccgcagtgcgtcgtccgctccaccccggccggctacctgctcgacccggcccactacgaactcgacagcgaccggttccggcacgcggtgctgaccgcccgggagttgcagcgggacgggcggctggcccaggcccgggcccgggtcgacgaggggctggcgctgtggcgcggccccgccctcggcgcggcggcgggcgccggactccagccccgggcccgccggctggaggaggaacgggtcttcgccctggagcagcgcgccgggctcgacctcgcgctcggccgccacgagacggccatcggcgaactcctcgacctcatcgcccagcatccgctgcgcgaggcggcctacgccgacctgatgctcgccctgtaccgttccggccgccagtccgacgcgctcgccgtctaccgcagggcgcagcgggtgctcgccgacgagctggccgtccgccccggcccccgcctcgccggcctggagcgggccatcctgcggcaggacgagtcgctgctggccggcgcggcggtgccctga

SEQ ID NO: 34 - DNA, 120 bp, Streptomyces viridochromogenes
  misc_feature: gene sequence of the aviRa promoter
tcaggggcctgcctccagcacgtcggctgcccggaccagtacggccgagcgggtgccgatcttcagccgctccagggcctttacgggagccaccgggatcttacggctgcggtcggtgac

SEQ ID NO: 35 - DNA, 621 bp, Streptomyces viridochromogenes
  misc_feature: gene sequence of the AviC1/AviC2 regulator
ctaggaacccgcggacgtatcgggtggatggtcggatccctctgcatcgccgatgtgtccgggaagcccgtgggcgaaggcaaccagtccggcctgaagacgggattcgaccccgagcttcgccagtatctgggccatatgagccttgacggtgcgctcggtgaccccgagcagcgcggcgatctcacggttggagtagccgtggctcagcaggaggaagacctggagctcgcggtcggagagtaaatgtacctggctgagcccttccagccaggggaactggtcctcgtggagaaatcgatcgtcgccagaatcactggaatcgcagccggaatatggcaaagtctggcccccgtatgagcgtgtggtccttgcatgccctaagaggtcatccgacgcatcgagtatcaaggcgccgaagggcgccaccactgaactatgaagacgtgagggcgataccacccatgcgacgaatgggtcctggacattactcatcttgatcatcttatcgcatctacggccgggttggggcgccttggtgccgcctgctgtcgtgagcagggcccgccgaggcgtgggcaaggcggataaggcggcccgtgcccggtgtgtgcacggcaa

SEQ ID NO: 36 - DNA, 130 bp, Nocardia uniformis
  misc_feature: gene sequence of the nocF promoter
catcacgaacctccagccgtgggatcgccctccggcagcatttatagacggtttgcttatcgatccgttttcacattcacccgcagtgataaggaattgataaacgattttcctagcctgagcggactat

SEQ ID NO: 37 - DNA, 1748 bp, Nocardia uniformis
  misc_feature: gene sequence of the NocR regulator
gtgcgcgcgggcgggcgccgggtccaggtcggcgggccgcgccagcggacggtgctggcgacgctgctgctcaacgccgaccgcgtggtgtcggtggacgcgctggccgagacggtctggggcgcccggcccccgtcgaccagccggacgcaggtggcgatctgcgtgtccgcgctgcgcaaggcgttccgcgcgagcggcgccgacgaggtgatcgagaccgtcgcgccggggtacgtcctgcgctccggcgggcaccggctggacaccctggacttcgacgaactggtggcgctggcgagggcggcggcccggcagggccggggcgcggaggccgtccggctgtacggctcggcgctcgcgctgcgccggggcccggtgctggcgaacgtgaccgggacggtgcccgagcacctgtcctgccagtgggaggagaccctgctcaccgcctacgaggagcaggtcgagctgcgcctggcgctgggcgagcaccgcctgctggtcgccgggctcgcggcggcggtcgagcggcacccgctgcgcgaccggctctacggcctgctcatcatcgcccagtaccgctccggccaccgggccgcggcgctggagacgttcgcccggttgcgccgccgctcggtcgacgagctcggcctggagccggggatggagctgcgccggctgcacgagcgcatcctgcgcgacgaggaccgcccggcggtcgagcgcccgccgtcgcagctgcccgccgcgacgcaggtgttcgtcgggcgcgccgaggagctggcggtgctggaccggctggccgccgaggacgggcaggcgggcgcgccgccgctcggactgctggtcggcggcgtcggcgtgggcaagaccgcgctggcggtgcggtgggcgcacgccaacgccgacctgttccccgacggccagctgttcgtcgacctgggcgggcacgacccgcaccacccgccgtcggcccccggcgccgtgctcgcgcacctgctgcacgcgctgggcgtgccgcccgagcgggtgccggtcgccgccgaacgacccgcgctgttccgcaccgcgatggccgcccgccggatgctgctggtgctggacgacgcccgcgacgcggcccaggtctggccgctgctgccgaacaccgccacctgccgggtgctggtgacctcccgcgacccgctgcgcgagctggtcgcccgcagcggggcggtgccgctgcggctgggcggcctcgggttcgacgagtccgtggcgctggtgcgcggcatcatcggcgaggcgcgggccgggcgcgacccggacgccctggtcgggctggtcgagctggtcgagctgtgcggtcgggtgccgggcgcgctgctggccgccgccgcgcacctggccagcaaaccgcactggggcgtgcccaggatggtccgggagctcaaccgcccgcgcagcaggctgtccggcctcggcgggcagcacctgcgcgacgggctcgcctccagcgcccgctgcctggacccggtggcggccgacctgtaccgggcgctgggcggcctgcccacgccggagctgacgtcctggacggccacggccctgctgggctgctcgacacccgaggccgacgacgtgctggagcgcctggtcgacgcgcacctgctggagcccgccggggcgggcgccggcggcgagagccactaccggctgcccagcctgtcccacgcctacgcggcgaacttgccacgaccggcccgtga

SEQ ID NO: 38 - DNA, 30 bp, Artificial
  Primer
cgcggatccctaagccgcaatccctgattg

SEQ ID NO: 39 - DNA, 35 bp, Artificial
  Primer
tccgatggacagtaaaagactggcccccaaagcag

SEQ ID NO: 40 - DNA, 46 bp, Artificial
  Primer
tgaggatccttattacttgtcagctcgtccatgccgagagtgatcc

SEQ ID NO: 41 - DNA, 55 bp, Artificial
  Primer
cttttactgtccatcggaactagctatggtgagcaagggcgaggagctgttcacc

SEQ ID NO: 42 - DNA, 22 bp, Artificial
  Primer
tcaactgctatcccccctgtta

SEQ ID NO: 43 - DNA, 33 bp, Artificial
  Primer
aaactcctttacttaaatgttttgataaataaa

SEQ ID NO: 44 - DNA, 22 bp, Artificial
  Primer
tacatatggtgagcaagggcga

SEQ ID NO: 45 - DNA, 30 bp, Artificial
  Primer
tagaattcttatctagacttgtacagctcg

SEQ ID NO: 46 - DNA, 24 bp, Artificial
  Primer
cggcgtttcacttctgagttcggc

SEQ ID NO: 47 - DNA, 30 bp, Artificial; identical to SEQ ID NO: 45
  Primer

SEQ ID NO: 48 - DNA, 1060 bp, Artificial
  gene construct
tcaactgctatcccccctgttattaaaacgcttacattgattattatagtcatttaattttaaatgtctatacttttataaaataaatataatcatatttttttccggttcaccgttttataaatttttctatggaagattcattcataatgtggtacactcatcaacggaaacgaatcaattaaatagctattatcacttgtataacctcaataatatggtttgagggtgtctaccaggaaccgtaaaatcctgattacaaaatttgtttatgacattttttgtaatcaggattttttttatttatcaaaacatttaagtaaaggagtttgttatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtctagataa

SEQ ID NO: 49 - DNA, 723 bp, Artificial
  synthetic fragment
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcaacttcaaggaggacggcaacatcctggggcacaagctggagtacaactacaacagccacaacgtctatatcatggccgacaagcagaagaacggcatcaaggtgaacttcaagatccgccacaacatcgagggcggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtctagataa

SEQ ID NO: 50 - DNA, 30 bp, Artificial
  Primer
gcgcggatcctcacacctgggggcgagctg

SEQ ID NO: 51 - DNA, 51 bp, Artificial
  Primer
gcgccatatgatatctccttcttaaagttcagcttgaatgaatctcttgcg

SEQ ID NO: 52 - DNA, 28 bp, Artificial
  Primer
gcgccatatggtgagcaagggcgaggag

SEQ ID NO: 53 - DNA, 34 bp, Artificial
  Primer
gcgcgtcgacttatctagacttgtacagctcgtc

SEQ ID NO: 54 - DNA, 20 bp, Artificial
  Primer
cgatcctgacgcagattttt

SEQ ID NO: 55 - DNA, 20 bp, Artificial
  Primer
ctcaccggctccagatttat

SEQ ID NO: 56 - DNA, 1765 bp, Artificial
  synthetic fragment
ggatccttattacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgccctcgatgttgtggcggatcttgaagttcaccttgatgccgttcttctgcttgtcggccatgatatagacgttgtggctgttgtagttgtactccagcttgtgccccaggatgttgccgtcctccttgaagttgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatatgatatctccttcttaaagttcatctaggtccgatggacagtaaaagactggcccccaaaagcagacctgtaatgaagatttccatgatcaccatcgtgacctatggaagtacttaagtaaaatgattggttcttaacatggtttaatatagcttcatgaaccccattcaactggacactttgctctcaatcattgatgaaggcagcttcgaaggcgcctccttagccctttccatttccccctcggcggtgagtcagcgcgttaaagctctcgagcatcacgtgggtcgagtgttggtatcgcgcacccaaccggccaaagcaaccgaagcgggtgaagtccttgtgcaagcagcgcggaaaatggtgttgctgcaagcagaaactaaagcgcaactatctggacgccttgctgaaatcccgttaaccatcgccatcaacgcagattcgctatccacatggtttcctcccgtgttcaacgaggtagcttcttggggtggagcaacgctcacgctgcgcttggaagatgaagcgcacacattatccttgctgcggcgtggagatgttttaggagcggtaacccgtgaagctaatcccgtggcgggatgtgaagtagtagaacttggaaccatgcgccacttggccattgcaaccccctcattgcgggatgcctacatggttgatgggaaactagattgggctgcgatgcccgtcttacgcttcggtcccaaagatgtgcttcaagaccgtgacctggacgggcgcgtcgatggtcctgtggggcgcaggcgcgtatccattgtcccgtcggcggaaggttttggtgaggcaattcgccgaggccttggttggggacttcttcccgaaacccaagctgctcccatgctaaaagcaggagaagtgatcctcctcgatgagatacccattgacacaccgatgtattggcaacgatggcgcctggaatctagatctctagctagactcacagacgccgtcgttgatgcagcaatcgagggattgcggccttaggtcgac

SEQ ID NO: 57 - DNA, 2506 bp, Artificial
  synthetic fragment
ggatcccgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcagtgggaattcctactggaacaggtggtggcgggcctcggcgcgctcgtactgctccaccacggtgtagtcctcgttgtgggaggtgatgtcgagcttgtagtccacgtagtggtagccgggcagcttcacgggcttcttggccatgtagatggacttgaactcacacaggtagtggccgccgcccttcagcttcagcgccatgtggttctcgcccttcagcacgccgtcgcgggggtagttgcgctcagtggagggctcccagcccagagtcttcttctgcattacggggccgtcggaggggaagttcacgccgatgaacttcacgtggtagatgagggtgccgtcctgcagggaggagtcctgggtcacggtcaccacgccgccgtcctcgaagttcatcacgcgctcccacttgaagccctcggggaaggactgcttgaggtagtcggggatgtcggcggggtgcttgatgtacgccttggagccgtagaagaactggggggacaggatgtcccaggcgaagggcagggggccgcccttggtcacttgcagcttggcggtctgggtgccctcgtagggcttgccctcgcccacgccctcgatctcgaactcgtggccgttcacggagccctccatgtgcaccttgaagcgcatgaagggcttgatgacgttctcagtgctatccatatgtatatctccttctgcaggcatgcaagcttggcgtaatcatggtcatatcttttaattctgtttcctgtgtgaaattgttatccgctcacaattccacacattatacgagccgatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgataagcccggatcagcttgcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgtcggccgccatgccggcgataatggcctgcttctcgccgaaacgtttggtggcgggaccagtgacgaaggcttgaggatcc

SEQ ID NO: 58 - DNA, 20 bp, Artificial
  Primer
gaacatcagcgacaggacaa

SEQ ID NO: 59 - DNA, 20 bp, Artificial
  Primer
gggaagcaaagaaacgaaca

SEQ ID NO: 60 - DNA, 20 bp, Artificial
  Primer
cctccccgggttgatattag

SEQ ID NO: 61 - DNA, 20 bp, Artificial
  Primer
ggccagcacgaatagcttta

SEQ ID NO: 62 - DNA, 20 bp, Artificial
  Primer
aggaatctccctgcgtacaa

SEQ ID NO: 63 - DNA, 20 bp, Artificial
  Primer
ccggattcatccaagaaagc

SEQ ID NO: 64 - DNA, 20 bp, Artificial
  Primer
gccttaaaacgccactcaat

SEQ ID NO: 65 - DNA, 20 bp, Artificial
  Primer
ggccgttgatcattgttctt

SEQ ID NO: 66 - DNA, 20 bp, Artificial
  Primer
aactccacgctggagctcac

SEQ ID NO: 67 - DNA, 17 bp, Artificial
  Primer
agaacgcggagtccacg

SEQ ID NO: 68 - protein, 521 aa, Artificial Sequence
  Amino acid sequence of murE L121F
MATTLLDLTKLIDGILKGSAQGVPAHAVGEQAIAAIGLDSSSLPTSDAIFAAVPGTRTHGAQFAGTDNAAKAVAILTDAAGLEVLNEAGETRPVIVVDDVRAVLGAASSSIYGDPSKDFTFIGVTGTSGKTTTSYLLEKGLMEAGHKVGLIGTTGTRIDGEEVPTKLTTPEAPTLQALFARMRDHGVTHVVMEVSSHALSLGRVAGSHFDVAAFTNLSQDHLDFHPTMDDYFDAKALFFRADSPLVADKQVVCVDDSWGQRMASVAADVQTVSTLGQEADFSATDINVSDSGAQSFKINAPSNQSYQVELALPGAFNVANATLAFAAAARVGVDGEAFARGMSKVAVPGRMERIDEGQDFLAVVDYAHKPAAVAAVLDTLRTQIDGRLGVVIGAGGDRDSTKRGPMGQLSAQRADLVIVTDDNPRSEVPATIRAAVTAGAQQGASESERPVEVLEIGDRAEAIRVLVEWAQPGDGIVVAGKGHEVGQLVAGVTHHFDDREEVRAALTEKLNNKLPLTTEEG

SEQ ID NO: 69 - DNA, 1566 bp, Artificial Sequence
  Nucleotide sequence of murE L121F
atggcaaccacgttgctggacctcaccaaacttatcgatggcatcctcaagggctctgcccagggcgttcccgctcacgcagtaggggaacaagcaatcgcggctattggtcttgactcctccagcttacctacctcggacgctatttttgctgcagttccaggaacccgcactcacggcgcacagtttgcaggtacggataacgctgcgaaagctgtggccattttgactgacgcagctggacttgaggtgctcaacgaagcaggagagacccgcccagtcatcgttgttgatgatgtccgcgcagtacttggcgcagcatcatcaagcatttatggcgatccttcaaaagatttcacgttcattggagtcactggaacctcaggtaaaaccaccaccagctacctcttggaaaaaggactcatggaggcaggccacaaagttggtttgatcggcaccacaggtacacgtattgacggggaagaagtacccacaaagctcaccactccagaagcgccgactctgcaggcattgtttgctcgaatgcgcgatcacggtgtcacccacgtggtgatggaagtatccagccatgcattgtcattgggcagagttgcgggttcccactttgatgtagctgcgtttaccaacctgtcgcaggatcaccttgatttccaccccaccatggatgattactttgacgcgaaggcattgttcttccgcgcagattctccacttgtggctgacaaacaggtcgtgtgcgtggatgattcttggggtcagcgcatggccagcgtggcagcggatgtgcaaacagtatccacccttgggcaagaagcagacttcagcgctacagacatcaatgtcagcgactctggcgcccagagttttaagatcaacgccccctcaaaccagtcctaccaggtcgagctagctcttccaggtgcgttcaacgttgctaacgccacgttggcatttgccgctgcggcacgcgtgggtgttgatggcgaagcgtttgctcgaggcatgtccaaggtcgcggttccaggccgtatggaacgcattgatgagggacaagacttccttgcagtggtggattatgcccacaagcctgctgcagtggctgctgtgttggatacgttgaggacccagattgacgggcgcctcggagtggttatcggtgctggtggagaccgcgattccaccaagcgtggccccatggggcagttgtccgcacagcgtgctgatctagttattgtcactgatgacaaccctcgttcagaggtgcctgccacgattcgcgcagcagtcactgcaggagcacagcagggtgcttcagagtccgaacgaccggtggaagtcctagaaattggtgaccgtgcagaagcaattcgcgttttggtcgagtgggcacagcctggagatggcattgtagtagctggaaaaggccatgaagttggacaactagttgctggtgtcacccaccattttgatgaccgcgaagaagttcgcgctgctttgacagaaaagctcaacaataaacttccccttactacggaagaaggatag

SEQ ID NO: 70 - DNA, 28 bp, Artificial
  Primer
taggatcccgacaacatcccactgtctg

SEQ ID NO: 71 - DNA, 27 bp, Artificial
  Primer
aagtcgacgtctgcttcttgcccaagg

SEQ ID NO: 72 - protein, 240 aa, Artificial Sequence
  Enhanced yellow fluorescence protein (eyfp)
VSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGINFKEDGNILGHKLEYNYNSHNVYIMADKQKNGIKVNFKIRHNIEGGSVQLADHYQQNTPIGDGPVLLPDNHYLSYQSALSKDPNEKRDHMVLLEFVTAAGITLGMDELYKSR

(19) United States
(12) Patent Application Publication
(10) Pub. No.: US 2016/0289776 A1
(43) Pub. Date: Oct. 6, 2016

(54) Sensors For The Detection Of Intracellular Metabolites

(71) Applicant: Forschungszentrum Jülich GmbH, Julich (DE)

(72) Inventors: Lothar Eggeling, Julich (DE); Michael Bott, Julich (DE); Stephan Binder, Eschweiler (DE); Julia Frunzke, Pulheim (DE); Nurije Mustafi, Dusseldorf (DE)

(21) Appl. No.: 15/147,177

(22) Filed: May 5, 2016

Related U.S. Application Data
(62) Division of application No. 13/695,769, filed on Feb. 28, 2013.

(30) Foreign Application Priority Data
May 3, 2010 (DE) 102010019059.4

(51) Int. Cl. C12Q 1/68; C12N 15/70; C12N 15/77

(52) U.S. Cl. CPC C12Q 1/6897; C12Q 1/689; C12N 15/77; C12N 15/70; C12Q 2600/156

(57) ABSTRACT

The present invention relates to a cell which is genetically modified with respect to its wild type and which comprises a gene sequence coding for an autofluorescent protein, wherein the expression of the autofluorescent protein depends on the intracellular concentration of a particular metabolite.

The present invention also relates to a method for the identification of a cell having an increased intracellular concentration of a particular metabolite, a method for the production of a cell which is genetically modified with respect to its wild type with optimized production of a particular metabolite, a cell obtained by this method, a method for the production of metabolites and a method for the preparation of a mixture.

### CROSS-REFERENCE TO RELATED APPLICATIONS

This is a divisional application of co-pending U.S. application Ser. No. 13/695,769, filed Nov. 1, 2012, which is the National Phase entry of International Application No. PCT/EP11/02196, filed May 3, 2011, which claims priority to German Patent Application No. 102010019059.4, filed May 3, 2010, the disclosures of which are incorporated herein by reference in their entireties.

### REFERENCE TO THE SEQUENCE LISTING

The Sequence Listing file identified as HFP0040-00US_ST25.txt, created Jun. 11, 2012, 51 KB, is incorporated herein by reference in its entirety.

### BACKGROUND

The present invention relates to a cell which is genetically modified with respect to its wild type, a method for the identification of a cell having an increased intracellular concentration of a particular metabolite, a method for the production of a cell which is genetically modified with respect to its wild type with optimized production of a particular metabolite, a cell obtained by this method, a method for the production of metabolites and a method for the preparation of a mixture.

Microbiologically produced metabolites are of great economic interest. Thus, amino acids, such as L-lysine, L-threonine, L-methionine and L-tryptophan, are used as a feedstuff additive, L-glutamate is used as a spice additive, L-isoleucine and L-tyrosine are used in the pharmaceuticals industry, L-arginine and L-isoleucine are used as a medicament or L-glutamate, L-aspartate and L-phenylalanine are used as a starting substance for the synthesis of fine chemicals. Another example of a metabolite which is relevant from the industrial point of view is oxoglutarate, which is used as a food supplement or as a precursor of arginine alpha-ketoglutarate, which promotes the release of growth hormones and insulin.

A preferred method for the production of such metabolites is the biotechnological production by means of microorganisms. In the production of amino acids in particular, the biologically active and optically active form of the particular metabolite can be obtained directly in this manner, and moreover simple and inexpensive raw materials can also be employed. Microorganisms which are employed are e.g. Corynebacterium glutamicum, its relatives ssp. flavum and ssp. lactofermentum (Liebl et al., Int. J System Bacteriol. 1991, 41: 255 to 260) or also Escherichia coli and related bacteria.

In the production of the metabolites described above by microbiological routes, regulation of the biosynthesis of the particular metabolite is conventionally modified by mutations such that they produce it beyond their own requirement and secrete it into the medium. Thus, for example, WO-A-2005/059139 discloses the production of L-lysine by means of a genetically modified Corynebacterium glutamicum strain, in which an increased L-lysine production is achieved by improving the metabolism via the pentose phosphate metabolic pathway. In WO-A-97/23597, an increase in the production of amino acids such as L-lysine in microorganisms is achieved by increasing the activity of export carriers which sluice these amino acids out of the cell.

Such over-producers are conventionally obtained by the search for mutants which produce the metabolites in a particularly large amount. This search is called “screening”. In the screening, random mutations (non-targeted mutagenesis) are induced in a starting strain, usually by means of conventional chemical or physical mutagens (e.g. MNNG or UV), and mutants are selected using conventional microbiological methods. Another possibility for providing metabolite over-producers comprises enhancing particular synthesis pathways by targeted gene over-expressions or deletions, or avoiding competing synthesis pathways.

The problem here, however, is that in the case of non-targeted mutagenesis in particular, in an accumulation of cells it is difficult to detect in which of the cells a mutation which has led to an increased intracellular synthesis of the metabolite in focus has taken place. The screening methods required for this are very time-consuming and costly.

### SUMMARY

The present invention was based on the object of overcoming the disadvantages resulting from the prior art in connection with the detection of genetically modified cells which over-produce a particular metabolite.

In particular, the present invention was based on the object of providing a genetically modified cell in which after a mutation those mutants which cause an over-production of a particular metabolite can be identified in a simple manner and optionally can be separated off from the remaining cells.

A further object on which the present invention was based consisted of providing a method for the identification of a cell having an increased intracellular concentration of a particular metabolite, which renders possible in a particularly simple and inexpensive manner an identification and optionally targeted separating off of such a cell in or from a large number of cells, for example in or from a cell suspension.

The present invention was also based on the object of providing a cell with optimized production of a particular metabolite in which genes or mutations which have been identified by the screening method described above as advantageous for an over-production of this metabolite are introduced in a targeted manner or produced by targeted mutations.

A contribution towards achieving the abovementioned objects is made by a cell which is genetically modified with respect to its wild type and which comprises a gene sequence coding for an autofluorescent protein, wherein the expression of the autofluorescent protein depends on the intracellular concentration of a particular metabolite.

The term “metabolite” as used herein is to be understood quite generally as meaning an intermediate product of a biochemical metabolic pathway, where according to the invention amine acids or amino acid derivatives, for example L-isoleucine, L-leucine, L-valine, L-lysine, L-arginine, L-citrulline, L-histidine, L-methionine, L-cysteine, L-tryptophan, L-glycine or O-acetyl-L-serine, nucleotides or nucleotide derivatives, for example xanthine, GTP or cyclic diguanosine monophosphate, fatty acids or fatty acid derivatives, for example acyl-coenzyme A thioesters, sugars or sugar derivatives, for example glucose, rhamnose, ribulose bis-phosphate, beta-D-galactosides or D-glucosamine 6-phosphate, keto acids, for example oxoglutarate, antibiotics, for example thienamycin, avilamycin, nocardicin or tetracyclines, vitamins or vitamin derivatives, for example biotin or thiamine pyrophosphate, or purine alkaloids, for example theophylline. “Derivatives” of the metabolites described above are understood as meaning in particular amines, phosphates or esters of the corresponding compounds. Very particularly preferred metabolites are amino acids, in particular an amino acid chosen from the group consisting of L-isoleucine, L-leucine, L-valine, L-lysine, L-arginine, L-citrulline, L-histidine, L-methionine, L-cysteine, L-tryptophan, O-acetyl-L-serine, particularly preferably from the group consisting of L-lysine, L-arginine, L-citrulline and L-histidine. The metabolite which is most preferred according to the invention is L-lysine.

A “wild type” of a cell is preferably understood as meaning a cell of which the genome is present in a state such as has formed naturally by evolution. The term is used both for the entire cell and for individual genes. In particular, those cells or those genes of which the gene sequences have been modified at least partly by humans by means of recombinant methods therefore do not fall under the term “wild type”.

Cells which are particularly preferred according to the invention are those of the genera Corynebacterium, Brevibacterium, Bacillus, Lactobacillus, Lactococcus, Candida, Pichia, Kluveromyces, Saccharomyces, Escherichia, Zymomonas, Yarrowia, Methylobacterium, Ralstonia and Clostridium, where Brevibacterium flavum, Brevibacterium lactofermentum, Escherichia coli, Saccharomyces cerevisiae, Kluveromyces lactis, Candida blankii, Candida rugosa, Corynebacterium glutamicum, Corynebacterium efficiens, Zymonomas mobilis, Yarrowia lipolytica, Methylobacterium extorquens, Ralstonia eutropha and Pichia pastoris are particularly preferred. Cells which are most preferred according to the invention are those of the genus Corynebacterium and Escherichia, where Corynebacterium glutamicum and Escherichia coli are very particularly preferred bacterial strains.

In the case in particular in which the metabolite is L-lysine, the cells which have been genetically modified can be derived in particular from cells chosen from the group consisting of Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869 and Brevibacterium divaricatum ATCC14020, and mutants and strains produced therefrom which produce L-amino acids, such as, for example, the L-lysine-producing strains Corynebacterium glutamicum PERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464 and Corynebacterium glutamicum DSM 5715 or such as, for example, the L-methionine-producing strain Corynebacterium glutamicum ATCC21608. Examples of suitable Escherichia coli strains which may be mentioned are Escherichia coli AJ11442 (see JP 56-18596 and U.S. Pat. No. 4,346,170), Escherichia coli strain VL611 and Escherichia coli strain WC196 (see WO-A-96/17930).

The cells according to the invention which are genetically modified with respect to their wild type are thus characterized in that they comprise a gene sequence coding for an autofluorescent protein, wherein the expression of this autofluorescent protein depends on the intracellular concentration of a particular metabolite.

All the gene sequences known to the person skilled in the art which code for an autofluorescent protein are possible as a gene sequence coding for an autofluorescent protein. Gene sequences which code for fluorescent proteins of the genus Aequora, such as green fluorescent protein (GFP), and variants thereof which are fluorescent in a different wavelength range (e.g. yellow fluorescent protein, YFP; blue fluorescent protein, BFP; cyan fluorescent protein, CFP) or of which the fluorescence is enhanced (enhanced GFP or EGFP, or EYFP, EBFP or ECFP), are particularly preferred. Gene sequences which code for other autofluorescent proteins, e.g., DsRed, HcRed, AsRed, AmCyan, ZsGreen, AcGFP, ZsYellow, such as are known from BD Biosciences, Franklin Lakes, USA, can furthermore also be used according to the invention.

The feature according to which the expression of the autofluorescent protein depends on the intracellular concentration of a particular metabolite and therefore can be controlled by the cell as a function of this metabolite concentration can thus be realized according to the invention in various manners and ways.

According to a first particular embodiment of the cell according to the invention, control of the expression of the gene sequence coding for the autofluorescent protein is effected as a function of the intracellular concentration of the particular metabolite at the transcription level. Depending on the intracellular concentration of the particular metabolite, more or less mRNA which can be translated in the ribosomes to form the autofluorescent proteins is consequently formed.

In connection with this first particular embodiment of the cell according to the invention, the control of the expression at the translation level can be effected by the gene sequence coding for the autofluorescent protein being under the control of a heterologous promoter which, in the wild type of the cell, controls the expression of a gene of which the expression in the wild-type cell depends on the intracellular concentration of a particular metabolite. The gene sequence coding for the autofluorescent protein can also be under the control of a promoter which is derived from such a promoter.

The wording “under the control of a heterologous promoter” indicates that the promoter in the natural manner, in particular in the wild-type cell from which the promoter sequence has been isolated and optionally genetically modified to further increase the promoter efficiency, does not regulate the expression of the gene sequence coding for the autofluorescent protein. In this connection, the wording “which is derived from such a promoter” means that the promoter which is contained in the genetically modified cell and regulates the expression of the gene sequence coding for the autofluorescent protein does not have to be a promoter which must be contained with an identical nucleic acid sequence in a wild-type cell. Rather, for the purpose of increasing the promoter efficiency, this promoter sequence can have been modified, for example, by insertion, deletion or exchange of individual bases, for example by palindromization of individual nucleic acid sequences. The promoter which regulates the expression of the gene sequence coding for the autofluorescent protein also does not necessarily have to be a promoter or derived from a promoter which is contained in the genome of the genetically modified cell itself. Nevertheless, it may prove to be entirely advantageous if the promoter is a promoter or is derived from a promoter which is contained in the genome of the genetically modified cell itself, but controls there the expression of a gene the expression of which depends on the intracellular concentration of a particular metabolite.

In this embodiment of the cell according to the invention, the gene sequence coding for the autofluorescent protein is under the control of a promoter. The term “under the control of a promoter” in this context is preferably to be understood as meaning that the gene sequence coding for the autofluorescent protein is functionally linked to the promoter. The promoter and the gene sequence coding for the autofluorescent protein are functionally linked if these two sequences and optionally further regulative elements, such as, for example, a terminator, are arranged sequentially such that each of the regulative elements can fulfil its function in the transgenic expression of the nucleic acid sequence. For this, a direct linking in the chemical sense is not absolutely necessary. Genetic control sequences, such as, for example, enhancer sequences, can also exert their function on the target sequence from further removed positions or even from other DNA molecules. Arrangements in which the gene sequence coding for the autofluorescent protein is positioned after the promoter sequence (i.e. at the 3′ end), so that the two sequences are bonded covalently to one another, are preferred. Preferably, in this context the distance between the gene sequence coding for the autofluorescent protein and the promoter sequence is less than 200 base pairs, particularly preferably less than 100 base pairs, very particularly preferably less than 50 base pairs. It is also possible for the gene sequence coding for the autofluorescent protein and the promoter to be linked functionally to one another such that there is still a part sequence of the homologous gene (that is to say that gene of which the expression in the wild-type cell is regulated by the promoter) between these two gene sequences. In the expression of such a DNA construct, a fusion protein from the autofluorescent protein and the amino acid sequence which is coded by the corresponding part sequence of the homologous gene is obtained. The lengths of such part sequences of the homologous gene are not critical as long as the functional capacity of the autofluorescent protein, that is to say its property of being fluorescent when excited with light of a particular wavelength, is not noticeably impaired.

In addition to the promoter and the gene sequence coding for the autofluorescent protein, according to this particular embodiment the cell according to the invention can also comprise a gene sequence coding for the regulator, wherein the regulator is preferably a protein which interacts in any manner with the metabolite and the promoter and in this manner influences the bonding affinity of the promoter sequence to the RNA polymerase. The interaction between the regulator and the promoter sequence in this context depends on the presence of the metabolite. As a rule, the metabolite is bound to particular, functional regions of the regulator and in this manner has the effect of a change in conformation of the regulator, which has an effect on the interaction between the regulator and the promoter sequence. In this context the regulator can in principle be an activator or a repressor.

According to the invention, possible promoters are in principle all promoters which usually control, via a functional linking, the expression of a gene of which the expression depends on the intracellular concentration of a particular metabolite. Very particularly preferably, the promoter is a promoter which usually controls the expression of a gene of which the expression depends on the intracellular concentration of a particular metabolite and which codes for a protein which renders possible the reduction of the intracellular concentration of a metabolite either via a chemical reaction of the metabolite or via the sluicing out of the metabolite from the cell. This protein is therefore either an enzyme which catalyses the reaction of the metabolite into a metabolism product which differs from the metabolite, or an active or passive transporter which catalyses the efflux of the metabolite from the cell.

The promoters can furthermore be those promoters which interact with particular activators in the presence of the metabolite and in this way cause expression of the gene sequence coding for the autofluorescent protein, or promoters which are inhibited by a repressor, the repressor diffusing away from the promoter by interaction with a particular metabolite, as a result of which the inhibition is eliminated and the expression of the gene sequence coding for the autofluorescent protein is effected.

Suitable examples of cells according to the invention of this first particular embodiment will now be described in more detail in the following. However, it is to be emphasized at this point that the present invention is not limited to the following examples which fall under the first particular embodiment of the cell according to the invention.

The genetically modified cell according to the first embodiment can thus be a genetically modified cell, preferably a genetically modified Pseudomonas putida cell, which comprises a gene sequence coding for an autofluorescent protein which is under the control of the bkd promoter (for the BkdR regulator in Pseudomonas putida see, for example, J. Bact., 181 (1999), pages 2,889-2,894, J. Bact., 187 (2005), page 664). An increased intracellular concentration of L-isoleucine, L-leucine, L-valine or D-leucine here leads to an expression of the autofluorescent protein. Such a cell preferably also contains, in addition to the bkd promoter and the gene sequence for an autofluorescent protein which is under the control of this promoter, a gene sequence coding for the BkdR regulator (branched-chain keto acid dehydrogenase regulatory protein). The DNA sequence of the bkd promoter regulated by the BkdR regulator is reproduced in SEQ ID NO:1, and the sequence of the BkdR regulator itself is reproduced in SEQ ID NO:2.

The genetically modified cell according to the first embodiment can furthermore be a genetically modified cell, preferably a genetically modified Bacillus subtilis cell, which comprises a gene sequence coding for an autofluorescent protein which is under the control of the ackA promoter (for the CodY repressor, see Mol. Mic. 62 (2006), page 811). Here also, an increased intracellular concentration of L-isoleucine, L-leucine and L-valine leads to an expression of the autofluorescent protein. Such a cell preferably also contains, in addition to the ackA promoter and the gene sequence for an autofluorescent protein which is under the control of this promoter, a gene sequence coding for the CodY repressor. The DNA sequence of the ackA promoter regulated by the CodY activator is reproduced in SEQ ID NO:3, and the sequence of the CodY activator itself is reproduced in SEQ ID NO:4.

The genetically modified cell according to the first embodiment can also be a genetically modified cell, preferably a genetically modified Pseudomonas putida cell, which comprises a gene sequence coding for an autofluorescent protein which is under the control of the mdeA promoter (for the MdeR regulator, see J. Bacteriol., 179 (1997), page 3,956). An increased intracellular concentration of L-methionine here leads to an expression of the autofluorescent protein. Such a cell preferably also contains, in addition to the mdeA promoter and the gene sequence for an autofluorescent protein which is under the control of this promoter, a gene sequence coding for the MdeR regulator. The DNA sequence of the mdeA promoter regulated by the MdeR regulator is reproduced in SEQ ID NO:5, and the sequence of the MdeR regulator itself is reproduced in SEQ ID NO:6.

The genetically modified cell according to the first embodiment can furthermore be a genetically modified cell, preferably a genetically modified Corynebacterium glutamicum cell, which comprises a gene sequence coding for an autofluorescent protein which is under the control of the brnF promoter (for the Lrp regulator in Corynebacterium glutamicum see J. Bact., 184 (14) (2002), pages 3,947-3,956). An increased intracellular concentration of L-isoleucine, L-leucine and L-valine here leads to an expression of the autofluorescent protein. Such a cell preferably also contains, in addition to the brnF promoter and the gene sequence for an autofluorescent protein which is under the control of this promoter, a gene sequence coding for the Lrp regulator. The DNA sequence of the brnF promoter regulated by the Lrp regulator is reproduced in SEQ ID NO:7, and the sequence of the Lrp regulator itself is reproduced in SEQ ID NO:8.

The genetically modified cell according to the first embodiment can furthermore be a genetically modified cell, preferably a genetically modified Escherichia coli cell, which comprises a gene sequence coding for an autofluorescent protein which is under the control of the cysP promoter (for the CysB regulator in Escherichia coli see Mol. Mic., 53 (2004), page 791). An increased intracellular concentration of O-acetyl-L-serine here leads to an expression of the autofluorescent protein. Such a cell preferably also contains, in addition to the cysP promoter and the gene sequence for an autofluorescent protein which is under the control of this promoter, a gene sequence coding for the CysB regulator. The DNA sequence of the cysP promoter regulated by the CysB regulator is reproduced in SEQ ID NO:9, and the sequence of the Lrp regulator itself is reproduced in SEQ ID NO:10.

The genetically modified cell according to the first embodiment can also be a genetically modified cell, preferably a genetically modified Escherichia coli cell, which comprises a gene sequence coding for an autofluorescent protein which is under the control of the cadB promoter (for the CadC regulator in Escherichia coli see Mol. Mic. 51 (2004), pages 1,401-1,412). An increased intracellular concentration of diamines such as cadaverine or putrescine here leads to an expression of the autofluorescent protein. Such a cell preferably also contains, in addition to the cadB promoter and the gene sequence for an autofluorescent protein which is under the control of this promoter, a gene sequence coding for the CadC regulator. The DNA sequence of the cadB promoter regulated by the CadC regulator is reproduced in SEQ ID NO:11, and the sequence of the CadC regulator itself is reproduced in SEQ ID NO:12.

The genetically modified cell according to the first embodiment can furthermore be a genetically modified cell, preferably a genetically modified Corynebacterium glutamicum cell, which comprises a gene sequence coding for an autofluorescent protein which is under the control of the metY, metK, horn, cysK, cysI or suuD promoter (for the McbR regulator in Corynebacterium glutamicum and the promoter sequences regulated by this see Mol. Mic. 56 (2005), pages 871-887). An increased intracellular concentration of S-adenosylhomocysteine here leads to an expression of the autofluorescent protein. Such a cell preferably also contains, in addition to the metY, metK, horn, cysK, cysI or suuD promoter and the gene sequence for an autofluorescent protein which is under the control of this promoter, a gene sequence coding for the McbR regulator. The DNA sequence of the metY promoter regulated by the McB regulator is reproduced in SEQ ID NO:13, and the sequence of the MecR regulator itself is reproduced in SEQ ID NO:14.

The genetically modified cell according to the first embodiment can also be a genetically modified cell, preferably a genetically modified Escherichia coli cell, which comprises a gene sequence coding for an autofluorescent protein which is under the control of the argO promoter. An increased intracellular concentration of L-lysine here leads to an expression of the autofluorescent protein. Such a cell preferably also contains, in addition to the argO promoter and the gene sequence for an autofluorescent protein which is under the control of this promoter, a gene sequence coding for the ArgP regulator. The DNA sequence of the argO promoter regulated by the ArgO regulator is reproduced in SEQ ID NO:15, and the sequence of the ArgP regulator itself is reproduced in SEQ ID NO:16.

The genetically modified cell according to a particularly preferred configuration of the first embodiment can moreover be a genetically modified cell, preferably a genetically modified Corynebacterium glutamicum cell, which comprises a gene sequence coding for an autofluorescent protein which is under the control of the lysE promoter (for the lysE promoter and its regulator LysG, see Microbiology, 147 (2001), page 1,765). An increased intracellular concentration of L-lysine, L-arginine, L-histidine and L-citrulline here leads to an expression of the autofluorescent protein. Such a cell preferably also contains, in addition to the lysE promoter and the gene sequence for an autofluorescent protein which is under the control of this promoter, a gene sequence coding for the LysG regulator. The DNA sequence of the lysE promoter regulated by the LysG regulator is reproduced in SEQ ID NO:17, and the sequence of the LysG regulator itself is reproduced in SEQ ID NO:18.

In Corynebacterium glutamicum the lysE gene codes for a secondary carrier which neither at the molecular nor at the structural level has similarities to one of the 12 known transporter superfamilies which are involved in the efflux of organic molecules and cations. On the basis of the novel function and unusual structure, LysE has been identified as the first member of a new translocator family. In the context of genome sequencings, it has since been possible to assign to this family numerous proteins, although hitherto still of largely unknown function. The LysE family to which LysE belongs forms, together with the RhtB family and the CadD family, the LysE superfamily, to which a total of 22 members are so far assigned. Of the LysE family, the lysine exporter from Corynebacterium glutamicum is so far the only functionally characteristic member. At the genetic level, lysE is regulated by the regulator LysG (governing L-lysine export). LysG has high similarities with bacterial regulator proteins of the LTTR family (LysR type transcriptional regulator). In this context, L-lysine acts as an inducer of the LysG-mediated transcription of lysE. In addition to L-lysine, the two basic amino acids L-arginine and L-histidine, as well as L-citrullline are also inducers of LysG-mediated lysE expression.

The genetically modified cell according to the first particular embodiment can furthermore be a genetically modified cell, preferably a genetically modified Escherichia coli cell, which comprises a gene sequence coding for an autofluorescent protein which is under the control of the fadE or fadBA promoter (for the FadR regulator in Escherichia coli see, for example, Mol. Biol., 29 (4) (2002), pages 937-943). An increased intracellular concentration of acyl-coenzyme A here leads to an expression of the autofluorescent protein. Such a cell preferably also contains, in addition to the fadE or fadBA promoter and the gene sequence for an autofluorescent protein which is under the control of this promoter, a gene sequence coding for the FadR regulator. The DNA sequence of the fadE promoter regulated by the FadR regulator is reproduced in SEQ ID NO:19, and the sequence of the LysG regulator itself is reproduced in SEQ ID NO:20.

The genetically modified cell according to the first particular embodiment can also be a genetically modified cell, preferably a genetically modified Bacillus subtilis cell, which comprises a gene sequence coding for an autofluorescent protein which is under the control of the fadM promoter (for the FabR regulator in Bacillus subtilis see, for example, J. Bacteriol., 191 (2009), pages 6,320-6,328). Here also, an increased intracellular concentration of acyl-coenzyme A leads to an expression of the autofluorescent protein. Such a cell preferably also contains, in addition to the fadM promoter and the gene sequence for an autofluorescent protein which is under the control of this promoter, a gene sequence coding for the FabR regulator. The DNA sequence of the fadM promoter regulated by the FabR regulator is reproduced in SEQ ID NO:21, and the sequence of the FabR regulator itself is reproduced in SEQ ID NO:22.

The genetically modified cell according to the first particular embodiment can furthermore be a genetically modified cell, preferably a genetically modified Escherichia coli cell, which comprises a gene sequence coding for an autofluorescent protein which is under the control of the rhaSR, rhaBAD or rhaT promoter (for the RhaR and RhaS regulator in Escherichia coli see, for example, J. Bacteriol., 189 (1) (2007), 269-271). An increased intracellular concentration of rhamnose here leads to an expression of the autofluorescent protein. Such a cell preferably also contains, in addition to the rhaSR, rhaBAD or rhaT promoter and the gene sequence for an autofluorescent protein which is under the control of this promoter, a gene sequence coding for the RhaR or RhaS regulator. The DNA sequence of the rhaSR promoter regulated by the RhaR regulator is reproduced in SEQ ID NO:23, the sequence of the rhaBAD promoter is reproduced in SEQ ID NO:24, the sequence of the RhaR regulator is reproduced in SEQ ID NO:25 and the sequence of the RhaS regulator is reproduced in SEQ ID NO:26.

The genetically modified cell according to the third configuration can also be a genetically modified cell, preferably a genetically modified Anabaena sp. cell, which comprises a gene sequence coding for an autofluorescent protein which is under the control of the hetC, nrrA or devB promoter (for the NtcA regulator in Anabaena sp. see, for example, J. Bacteriol., 190 (18) (2008), pages 6,126-6,133). An increased intracellular concentration of oxoglutarate here leads to an expression of the autofluorescent protein. Such a cell preferably also contains, in addition to the hetC, nrrA or devB promoter and the gene sequence for an autofluorescent protein which is under the control of this promoter, a gene sequence coding for the NtcA regulator. The DNA sequence of the hetC promoter regulated by the NtcA regulator is reproduced in SEQ ID NO:27, the sequence of the nrrA promoter is reproduced in SEQ ID NO:28, the sequence of the devB promoter is reproduced in SEQ ID NO:29 and the sequence of the NtcA regulator is reproduced in SEQ ID NO:30.

The genetically modified cell according to the first particular embodiment can furthermore be a genetically modified cell, preferably a genetically modified Mycobacterium sp. cell, which comprises a gene sequence coding for an autofluorescent protein which is under the control of the cbbLS-2 or cbbLS-1 promoter (for the CbbR regulator in Mycobacterium sp. see, for example, Mol. Micr. 47 (2009), page 297). An increased intracellular concentration of ribulose bis-phosphate here leads to an expression of the autofluorescent protein. Such a cell preferably also contains, in addition to the cbbLS-2 or cbbLS-1 promoter and the gene sequence for an autofluorescent protein which is under the control of this promoter, a gene sequence coding for the CbbR regulator. The DNA sequence of the CbbR regulator is reproduced in SEQ ID NO:31.

The genetically modified cell according to the first particular embodiment can furthermore be a genetically modified cell, preferably a genetically modified Streptomyces cattleya cell, which comprises a gene sequence coding for an autofluorescent protein which is under the control of the pcbAB promoter (for the ThnU regulator in Streptomyces cattleya see, for example, Mol. Micr., 69 (2008), page 633). An increased intracellular concentration of thienamycin here leads to an expression of the autofluorescent protein. Such a cell preferably also contains, in addition to the pcbA promoter and the gene sequence for an autofluorescent protein which is under the control of this promoter, a gene sequence coding for the ThnU regulator. The DNA sequence of the pcbAB promoter regulated by the ThnU regulator is reproduced in SEQ ID NO:32, and the sequence of the ThnU regulator itself is reproduced in SEQ ID NO:33.

The genetically modified cell according to the first particular embodiment can also be a genetically modified cell, preferably a genetically modified Streptomyces viridochromogenes cell, which comprises a gene sequence coding for an autofluorescent protein which is under the control of the aviRa promoter (for the AviC1 or AviC2 regulator in Streptomyces viridochromogenes see, for example, J. Antibiotics, 62 (2009), page 461). An increased intracellular concentration of avilamycin here leads to an expression of the autofluorescent protein. Such a cell preferably also contains, in addition to the aviRa promoter and the gene sequence for an autofluorescent protein which is under the control of this promoter, a gene sequence coding for the AviC1 and/or AviC2 regulator. The DNA sequence of the aviRa promoter regulated by the AviC1 or AviC2 regulator is reproduced in SEQ ID NO:34, and the sequence of the AviC1 or AviC2 regulator itself is reproduced in SEQ ID NO:35.

The genetically modified cell according to the first particular embodiment can furthermore be a genetically modified cell, preferably a genetically modified Nocardia uniformis cell, which comprises a gene sequence coding for an autofluorescent protein which is under the control of the nocF promoter (for the NocR regulator in Nocardia uniformis see, for example, J. Bacteriol., 191 (2009), page 1,066). An increased intracellular concentration of nocardicin here leads to an expression of the autofluorescent protein. Such a cell preferably also contains, in addition to the nocF promoter and the gene sequence for an autofluorescent protein which is under the control of this promoter, a gene sequence coding for the NocR regulator. The DNA sequence of the nocF promoter regulated by the NocR regulator is reproduced in SEQ ID NO:36, and the sequence of the NocR regulator itself is reproduced in SEQ ID NO:37.

In principle there are thus various possibilities for producing a cell according to the invention according to the first particular embodiment comprising a promoter described above and a nucleic acid which codes for an autofluorescent protein and is under the control of this promoter.

A first possibility consists of, for example, starting from a cell of which the genome already comprises one of the promoters described above and preferably a gene sequence coding for the corresponding regulator, and then introducing into the genome of the cell a gene sequence coding for an autofluorescent protein such that this gene sequence is under the control of the promoter. If appropriate, the nucleic acid sequence of the promoter itself can be modified, before or after the integration of the gene sequence coding for the autofluorescent protein into the genome, by one or more nucleotide exchanges, nucleotide deletions or nucleotide insertions for the purpose of increasing the promoter efficiency.

A second possibility consists, for example, of introducing into the cell one or more nucleic acid constructs comprising the promoter sequence and the gene sequence which codes for the autofluorescent protein and is under the control of the promoter, it also being possible here to modify the nucleic acid sequence of the promoter itself by one or more nucleotide exchanges, nucleotide deletions or nucleotide insertions for the purpose of increasing the promoter efficiency. The insertion of the nucleic acid construct can take place chromosomally or extrachromosomally, for example on an extrachromosomally replicating vector. Suitable vectors are those which are replicated in the particular bacteria strains. Numerous known plasmid vectors, such as e.g. pZ1 (Menkel et al., Applied and Environmental Microbiology (1989) 64: 549-554), pEKE×1 (Eikmanns et al., Gene 102: 93-98 (1991)) or pHS2-1 (Sonnen et al., Gene 107: 69-74 (1991)) are based on the cryptic plasmids pHM1519, pBL1 or pGA1. Other plasmid vectors, such as e.g. those which are based on pCG4 (U.S. Pat. No. 4,489,160), or pNG2 (Serwold-Davis et al., FEMS Microbiology Letters 66, 119-124 (1990)), or pAG1 (U.S. Pat. No. 5,158,891), can be used in the same manner. However, this list is not limiting for the present invention.

Instructions for the production of gene constructs comprising a promoter and a gene sequence under the control of this promoter and the sluicing of such a construct into the chromosome of a cell or the sluicing of an extrachromosomally replicating vector comprising this gene construct into a cell are sufficiently known to the person skilled in the art, for example from Martin et al. (Bio/Technology 5, 137-146 (1987)), from Guerrero et al. (Gene 138, 35-41 (1994)), from Tsuchiya and Morinaga (Bio/Technology 6, 428-430 (1988)), from Eikmanns et al. (Gene 102, 93-98 (1991)), from EP-A-0 472 869, from U.S. Pat. No. 4,601,893, from Schwarzer and Paler (Bio/Technology 9, 84-87 (1991), from Remscheid et al. (Applied and Environmental Microbiology 60, 126-132 (1994)), from LaBarre et al. (Journal of Bacteriology 175, 1001-1007 (1993)), from WO-A-96/15246, from Malumbres et al. (Gene 134, 15-24 (1993), from JP-A-10-229891, from Jensen and Hammer (Biotechnology and Bioengineering 58, 191-195 (1998)) and from known textbooks of genetics and molecular biology.

According to a second particular embodiment of the cell according to the invention, control of the expression of the gene sequence coding for the autofluorescent protein is effected as a function of the intracellular concentration of the particular metabolite by means of a so-called “riboswitch” it being possible for the expression to be regulated by means of such a “riboswitch” both at the transcription level and at the translation level. A “riboswitch” is understood as meaning regulatory elements which consist exclusively of mRNA. They act as a sensor and as a regulatory element at the same time. An overview of riboswitches is to be found, for example, in Vitrechak et al., Trends in Genetics, 20 (1) (2004), pages 44-50. Further details on regulation of gene expression with a riboswitch can also be found in the dissertation by Jonas Noeske (2007) entitled “Strukturelle Untersuchungen an Metabolit-bindenden Riboswitch-RNAs mittels NMR”, submitted to the Faculty of Biochemistry, Chemistry and Pharmacy of the Johann Wolfgang Goethe University in Frankfurt am Main.

Riboswitches can be used in the cells according to the invention according to this second particular embodiment in that the gene sequence coding for the autofluorescent protein is bonded functionally to a DNA sequence which is capable of binding the metabolite at the mRNA level, either the further transcription along the DNA or the translation on the ribosomes being influenced as a function of the binding of the metabolite to the mRNA. The expression of the gene sequence coding for the autofluorescent protein is regulated by the riboswitch at the transcription level or the translation level in this manner. In the cells according to the invention with riboswitch elements, the metabolite is bound directly to a structured region in the 5′-UTR of the mRNA without the involvement of any protein factors, and induces a change in the RNA secondary structure. This change in conformation in the 5′-UTR leads to modulation of the expression of the following gene coding for the autofluorescent protein. In this context, the gene-regulating action can be achieved by influencing either the transcription or the translation, or if appropriate also the RNA processing. The metabolite-binding region of the riboswitches (aptamer domain) is a modular, independent RNA domain. The remaining part of the riboswitch (expression platform) usually lies downstream of the aptamer domain. Depending on whether a metabolite is bound to the aptamer domain or not, the expression platform can enter into base pairings with regions of the aptamer domain. In most cases these base pairings between the expression platform and the aptamer domain take place in the non-bound metabolite state and lead to activation of the gene expression. Conversely, these base pairings are impeded in the ligand-bound state, which usually leads to inhibition of gene expression. Whether the regulation mechanism has an effect on the transcription or the translation depends on the secondary structure which the expression platform assumes in the metabolite-bound or non-bound metabolite state. The expression platform often contains sequences which can form a transcription terminator and a transcription antiterminator, the two secondary structures, however, being mutually exclusive. Another motif which frequently occurs is a secondary structure by which the SD sequence (Shine-Dalgarno sequence) is converted into a single-stranded form or masked, depending on the metabolite binding state. If the SD sequence is masked by formation of a secondary structure, the SD sequence cannot be recognized by the ribosome. Premature discontinuation of transcription or the initiation of translation can be regulated by riboswitches in this manner.

Examples which may be mentioned of suitable riboswitch elements which render possible control of the expression of the autofluorescent protein at the transcription level or the translation level are, for example, the lysine riboswitch from Bacillus subtilis (described by Grundy et al., 2009), the glycine riboswitch from Bacillus subtilis (described by Mandal et al., Science 306 (2004), pages 275-279), the adenine riboswitch from Bacillus subtilis (described by Mandal and Breaker, Nat. Struct. Mol. Biol. 11 (2004), pages 29-35) or the TPP tandem riboswitch from Bacillus anthracia (described by Welz and Breaker, RNA 13 (2007), pages 573-582). In addition to these naturally occurring riboswitch elements, synthetic riboswitch elements can also be used, such as, for example, the theophylline riboswitch (described by Jenison et al., Science 263 (1994), pages 1,425-1,429 or by Desai and Gellivan, J. Am. Chem. Soc. 126 (2004), pages 1.3247-54), the biotin riboswitch (described by Wilson et al., Biochemistry 37 (1998), pages 14,410-14,419) or the Tet riboswitch (described by Berens et al., Bioorg. Med. Chem. 9 (2001), pages 2,549-2,556).

A contribution towards achieving the abovementioned objects is furthermore made by a method for the identification of a cell having an increased intracellular concentration of a particular metabolite in a cell suspension, comprising the method steps:
i) provision of a cell suspension comprising the cells according to the invention described above which are genetically modified with respect to their wild type and which comprise a gene sequence coding for an autofluorescent protein, wherein the expression of the autofluorescent protein depends on the intracellular concentration of a particular metabolite;
ii) genetic modification of the cells to obtain a cell suspension in which the cells differ with respect to the intracellular concentration of a particular metabolite;
iii) identification of individual cells in the cell suspension having an increased intracellular concentration of this particular metabolite by detection of the intracellular fluorescence activity.

In step i) of the method according to the invention, a cell suspension comprising a nutrient medium and a large number of the genetically modified cells described above is first provided.

In step ii) of the method according to the invention one or more of the cells in the cell suspension is or are then genetically modified in order to obtain a cell suspension in which the cells differ with respect to the intracellular concentration of a particular metabolite.

The genetic modification of the cell suspension can be carried out by targeted or non-targeted mutagenesis, non-targeted mutagenesis being particularly preferred.

In targeted mutagenesis, mutations are generated in particular genes of the cell in a controlled manner Possible mutations are transitions, transversions, insertions and deletions. Depending on the effect of the amino acid exchange on the enzyme activity, “missense mutations” or “nonsense mutations” are referred to. Insertions or deletions of at least one base pair in a gene lead to “frame shift mutations”, as a consequence of which incorrect amino acid are incorporated or the translation is discontinued prematurely. Deletions of several codons typically lead to a complete loss of the enzyme activity. Instructions for generating such mutations belong to the prior art and can be found in known textbooks of genetics and molecular biology, such as e.g. the textbook by Knippers (“Molekulare Genetik”, 6th edition, Georg Thieme-Verlag, Stuttgart, Germany, 1995), that by Winnacker (“Gene and Klone”, VCH Verlagsgesellschaft, Weinheim, Germany, 1990) or that by Hagemann (“Allgemeine Genetik”, Gustav Fischer-Verlag, Stuttgart, 1986).

Details, in particular helpful literature references relating to these methods of targeted mutagenesis, can be found, for example, in DE-A-102 24 088.

However, it is particularly preferable according to the invention if the genetic modification in method step ii) is carried out by non-targeted mutagenesis. An example of such a non-targeted mutagenesis is treatment of the cells with chemicals such as e.g. N-methyl-N-nitro-N-nitrosoguanidine or irradiation of the cells with UV light. Such methods for inducing mutations are generally known and can be looked up, inter alia, in Miller (“A Short Course in Bacterial Genetics, A Laboratory Manual and Handbook for Escherichia coli and Related Bacteria” (Cold Spring Harbor Laboratory Press, 1992)) or in the handbook “Manual of Methods for General Bacteriology” of the American Society for Bacteriology (Washington D.C., USA, 1981).

By the genetic modification of the cell in method step ii), depending on the nature of the mutation which has taken place in the cell, in a particular cell, for example as a consequence of an increased or reduced enzyme activity, an increased or reduced expression of a particular enzyme, an increased or reduced activity of a particular transporter protein, an increased or reduced expression of a particular transporter protein, a mutation in a regulator protein, a mutation in a structure protein or a mutation in an RNA control element, there may be an increase in the intracellular concentration of that metabolite which has an influence on the expression of the autofluorescent protein by interaction with a corresponding regulator protein via the promoter or by interaction with a riboswitch element. A cell in which the concentration of a particular metabolite is increased as a consequence of the mutation is therefore distinguished in that the autofluorescent protein is formed in this cell. The gene for the autofluorescent protein thus acts as a reporter gene for an increased intracellular metabolite concentration.

In method step iii) of the method according to the invention, individual cells in the cell suspension having an increased intracellular concentration of this particular metabolite are therefore identified by detection of the intracellular fluorescence activity. For this, the cell suspension is exposed to electromagnetic radiation in that frequency which excites the autofluorescent proteins to emission of light.

According to a particular configuration of the method according to the invention, after, preferably directly after the identification of the cells in method step iii), a further method step iv) is carried out, in which the cells identified are separated off from the cell suspension, this separating off preferably being carried out by means of flow cytometry (FACS=fluorescence activated cell sorting), very particularly preferably by means of high performance flow cytometry (HAT-FACS=high throughput fluorescence activated cell sorting). Details on the analysis of cell suspensions by means of flow cytometry can be found, for example, in Sack U, Tarnok A, Rothe G (eds.): Zelluläre Diagnostik. Grundlagen, Methoden and klinische Anwendungen der Durchflusszytometrie, Basel, Karger, 2007, pages 27-70.

By means of the method according to the invention, in a cell suspension in which targeted or non-targeted mutations have been generated in the cells it is therefore possible to isolate in a targeted manner, without influencing the vitality of the cells, those cells in which the mutation has led to an increased intracellular concentration of a particular metabolite.

A contribution towards achieving the abovementioned objects is also made by a method for the production of a cell which is genetically modified with respect to its wild type with optimized production of a particular metabolite, comprising the method steps:
I) provision of a cell suspension comprising the cells according to the invention described above which are genetically modified with respect to their wild type and which comprise a gene sequence coding for an autofluorescent protein, wherein the expression of the autofluorescent protein depends on the intracellular concentration of a particular metabolite;
II) genetic modification of the cells to obtain a cell suspension in which the cells differ with respect to their intracellular concentration of a particular metabolite;
III) identification of individual cells in the cell suspension having an increased intracellular concentration of the particular metabolite by detection of the intracellular fluorescence activity.
IV) separating off of the identified cells from the cell suspension;
V) identification of those genetically modified genes G₁ to Gₙ or those mutations M₁ to Mₘ in the cells identified and separated off which are responsible for the increased intracellular concentration of the particular metabolite;
VI) production of a cell which is genetically modified with respect to its wild type with optimized production of the particular metabolite, of which the genome comprises at least one of the genes G₁ to Gₙ and/or at least one of the mutations M₁ to Mₘ.

According to method steps I) to IV), cells having an increased intracellular concentration of a particular metabolite are first generated by mutagenesis and are separated off from a cell suspension, it being possible to refer here to method steps i) to iv) described above.

In method step V), in the cells identified and separated off, those genetically modified genes G₁ to Gₙ or those mutations M₁ to Mₘ which are responsible for the increased intracellular concentration of the particular metabolite are then identified by means of genetic methods known to the person skilled in the art, the numerical value of n and m depending on the number of modified genes observed and, respectively of mutations observed in the cell identified and separated off. Preferably, the procedure in this context is such that the sequence of those genes or promoter sequences in the cells which are known to stimulate the formation of a particular metabolite is first analysed. In the case of L-lysine as the metabolite, these are, for example, the genes lysC, horn, zwf, mqo, leuC, gnd or pyk. If no mutation is recognized in any of these genes, the entire genome of the cell identified and separated off is analysed in order to identify, where appropriate, further modified genes Gᵢ or further mutations Mᵢ. Advantageous modified gene sequences Gᵢ or advantageous mutations Mᵢ which lead to an increase in the intracellular concentration of a particular metabolite in a cell can be identified in this manner.

In a further method step VI), a cell which is genetically modified with respect to its wild type with optimized production of the particular metabolite, of which the genome comprises at least one of the genes G₁ to Gₙ and/or at least one of the mutations M₁ to Mₘ can then be produced. For this, one or more of the advantageous modified genes G and/or modified mutations M observed in method step V) are introduced into a cell in a targeted manner. This targeted introduction of particular mutations can be carried out, for example, by means of “gene replacement”. In this method, a mutation, such as e.g. a deletion, insertion or base exchange, is produced in vitro in the gene of interest. The allele produced is in turn cloned into a vector which is non-replicative for the target host and this is then transferred into the target host by transformation or conjugation. After homologous recombination by means of a first “cross-over” event effecting integration and a suitable second “cross-over” event effecting an excision in the target gene or in the target sequence, the incorporation of the mutation or the allele is achieved.

A contribution towards achieving the abovementioned objects is also made by a cell with optimized production of a particular metabolite which has been obtained by the method described above.

A contribution towards achieving the abovementioned objects is also made by a process for the production of metabolites, comprising the method steps:
(a) production of a cell which is genetically modified with respect to its wild type with optimized production of a particular metabolite by the method described above;
(b) cultivation of the cell in a culture medium comprising nutrients under conditions under which the cell produces the particular metabolite from the nutrients.

The genetically modified cells according to the invention with optimized production of a particular metabolite which are produced in method step (a) can be cultivated in the nutrient medium in method step (b) continuously or discontinuously in the batch method (batch cultivation) or in the fed batch method (feed method) or repeated fed batch method (repetitive feed method) for the purpose of production of the metabolite. A semi-continuous method such as is described in GB-A-1009370 is also conceivable. A summary of known cultivation methods is described in the textbook by Chmiel (“Bioprozesstechnik 1. Einführung in die Bioverfahrenstechnik” (Gustav Fischer Verlag, Stuttgart, 1991)) or in the textbook by Storhas (“Bioreaktoren and periphere Einrichtungen”, Vieweg Verlag, Braunschweig/Wiesbaden, 1994).

The nutrient medium to be used must meet the requirements of the particular strains in a suitable manner Descriptions of culture media of various microorganisms are contained in the handbook “Manual of Methods for General Bacteriology” of the American Society for Bacteriology (Washington D.C., USA, 1981).

The nutrient medium can comprise carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and methanol, hydrocarbons, such as methane, amino acids, such as L-glutamate or L-valine, or organic acids, such as e.g. acetic acid, as a source of carbon. These substances can be used individually or as a mixture.

The nutrient medium can comprise organic nitrogen-containing compounds, such as peptones, yeast extract, meat extract, malt extract, corn steep liquor, soya bean flour and urea, or inorganic compounds, such as ammonium sulphate, ammonium chloride, ammonium phosphate, ammonium carbonate and ammonium nitrate, as a source of nitrogen. The sources of nitrogen can be used individually or as a mixture.

The nutrient medium can comprise phosphoric acid, potassium dihydrogen phosphate or dipotassium hydrogen phosphate or the corresponding sodium-containing salts as a source of phosphorus. The nutrient medium must furthermore comprise salts of metals, such as e.g. magnesium sulphate or iron sulphate, which are necessary for growth. Finally, essential growth substances, such as amino acids and vitamins, can be employed in addition to the abovementioned substances. Suitable precursors can moreover be added to the nutrient medium. The starting substances mentioned can be added to the culture in the form of a one-off batch or can be fed in during the cultivation in a suitable manner.

Basic compounds, such as sodium hydroxide, potassium hydroxide, ammonia or aqueous ammonia, or acidic compounds, such as phosphoric acid or sulphuric acid, are employed in a suitable manner to control the pH of the culture. Antifoam agents, such as e.g. fatty acid polyglycol esters, can be employed to control the development of foam. Suitable substances having a selective action, such as e.g. antibiotics, can be added to the medium to maintain the stability of plasmids. Oxygen or oxygen-containing gas mixtures, such as e.g. air, are introduced into the culture in order to maintain aerobic conditions. The temperature of the culture is usually 20° C. to 45° C., and preferably 25° C. to 40° C.

A contribution towards achieving the abovementioned objects is also made by a method for the preparation of a mixture comprising the method steps:
(A) production of metabolites by the method described above;
(B) mixing of the metabolite with a mixture component which differs from the metabolite.

If the metabolite is an amino acid, in particular L-lysine, the mixture is preferably a foodstuff, very particularly preferably an animal feed, or a pharmaceutical composition.

The invention is now explained in more detail with the aid of figures and non-limiting examples.

### DESCRIPTION OF THE DRAWINGS

FIG. 1 shows possible constructs in which the gene sequence of an autofluorescent protein (afp) according to the first embodiment of the cell according to the invention is under the control of a promoter (lysE promoter).

FIG. 2 shows the vector pJC1lysGE′eYFP produced in Example 1 (lysE′eYFP, coding sequence of the LysE′eYFP fusion protein; lysG, coding sequence of the regulator protein LysG; kanR, coding sequence of the kanamycin-mediated resistance; repA: replication origin; BamHI: recognition sequence and cleavage site of the restriction enzyme BamHI).

FIG. 3 shows a confocal microscope image of the strains ATCC 13032 pJC1lysGE′eYFP (top) and DM1800 p JC1lysGE′eYFP (bottom) obtained in Example 1. The white bar in the lower image corresponds to a length of 10 μm. In each case 3 μl of cell suspensions were placed on a slide and immobilized by a thin layer of 1 agarose. The immobilized suspension was excited with light of wavelength 514 nm and an exposure time of 700 ms. The fluorescence emission measurement of eYFP was carried out with a Zeiss AxioImager M1 using a broadband filter in the range of from 505 nm to 550 nm.

FIG. 4 shows the sequence of the gene sequence produced in Example 2 based on a riboswitch element, comprising a riboswitch element and a gene sequence linked functionally to this riboswitch element and coding for an autofluorescent protein (bold: aptamer; italics: terminator sequence; underlined: EYFP).

FIG. 5 shows the vector pJC1lrp-brnF′eYFP.

FIG. 6 shows the correlation of the internal L-methionine concentration with the fluorescence output signal of the ATCC13032pJC1lrp-brnF′-eYFP cultures obtained in Example 3.

FIG. 7 shows the formation of lysine by the mutants of the starting strain ATCC13032pSenLysTK-C in Example 4c).

### DETAILED DESCRIPTION

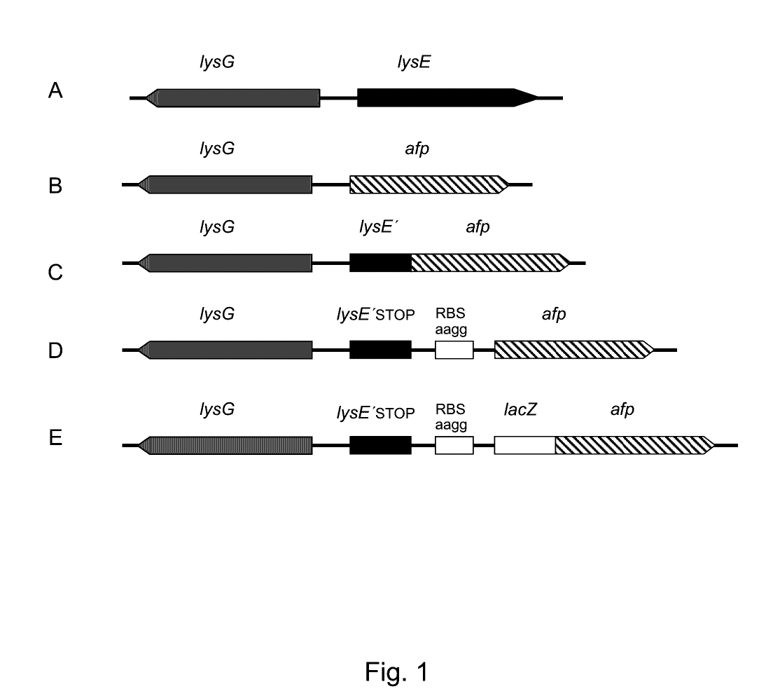

FIG. 1 shows possible constructs in which the gene sequence of an autofluorescent protein (afp) according to the first embodiment of the cell according to the invention is under the control of a promoter (lysE promoter). Variant A indicates a starting situation in which the metabolite-dependent regulator lies directly adjacent to its target gene (lysE), which it regulates according to the metabolite concentration. According to variant B, in the simplest case the target gene is replaced by a fluorescent protein (afp). According to variant C, a translational fusion of the first amino acids of the target gene with the fluorescent protein has taken place. In variant D, a transcriptional fusion has taken place such that a long transcript is formed, starting from the promoter region which comprises the first amino acids of the target gene and ending by a stop codon, followed by a ribosome-binding site (RBS) and the open reading frame for the fluorescent protein. In variant E, a transcriptional fusion has taken place such that a long transcript is formed, starting from the promoter region which comprises the first amino acids of the target gene and ending by a stop codon, followed by a ribosome-binding site and the start of a known and well-expressed protein, such as e.g. the beta-galactosidase from E. coli, LacZ, which in turn is fused with the fluorescent protein.

### EXAMPLES

### Example 1

Production of a cell according to the invention according to the first embodiment by the example of a cell in which a gene sequence coding for an autofluorescent protein is under the control of the lysE promoter and in which the expression of the autofluorescent protein depends on the intracellular L-lysine concentration.

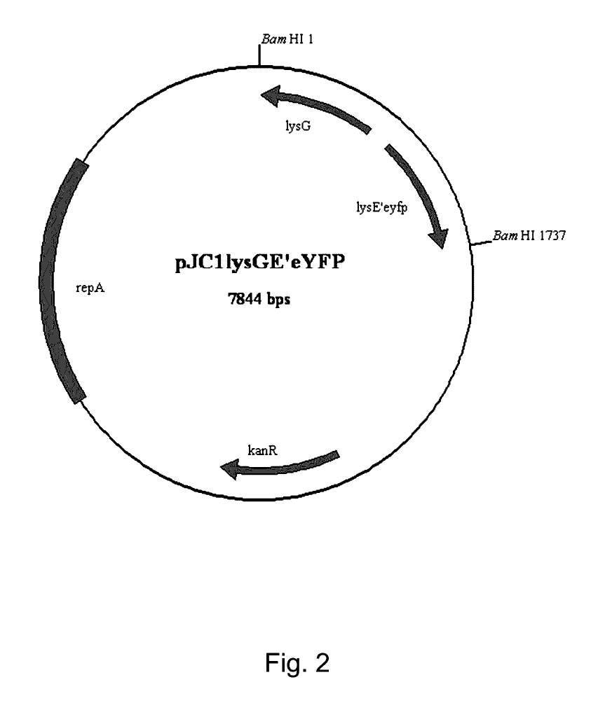

a) Construction of the Vector pJC1lysGE′eYFP (FIG. 2)

The construction of the fusion of lysE′ with the reporter gene eyfp (SEQ ID NO:49; protein sequence of the eYFP: SEQ ID NO:72) was achieved by an overlap extension PCR. pUC18-2.3-kb-lysGE-BamHI, which carries the coding sequence of lysE together with the gene of the divergently transcribed regulator LysG (Bellmann et al., 2001; Microbiology 1471765-74), and pEKE×2-yfp-tetR (Frunzke et al., 2008; J Bacteriol. 190:5111-9), which renders possible amplification of eyfp, served as templates. To establish the lysGE′eyfp fragment, the coding sequences lysGE′ and lysGE′ns (1,010 bp) were first amplified with the oligonucleotide combinations plysGE_for (SEQ ID NO:38) and plysGE_rev (SEQ ID NO:39). For amplification of the coding sequence of eyfp, the two oligonucleotide combinations peYFP_rev (SEQ ID NO:40) and peYFP_fw2 (SEQ ID NO:41) were used.

plysGE_for
(SEQ ID NO: 38)
5′-CGCGGATCCCTAAGCCGCAATCCCTGATTG-3′
plysGE_rev
(SEQ ID NO: 39)
5′-TCCGATGGACAGTAAAAGACTGGCCCCCAAAGCAG-3′
peYFP_rev
(SEQ ID NO: 40)
5′-TGAGGATCCTTATTACTTGTCAGCTCGTCCATGCCGAG
AGTGATCC-3′
peYFP_fw2
(SEQ ID NO: 41)
5′-CTTTTACTGTCCATCGGAACTAGCTATGGTGAGCAAGG
GCGAGGAGCTGTTCACC-3′

After purification of the amplified fragments from a 1% strength agarose gel, these were employed as matrices in a second PCR reaction with the outer primers plysGE-for and peYFP_rev. By hybridization of the template fragments in a complementary region of 17 bp created from the inner oligonucleotide primers plysGE_rev and peYFP_fw2, it was possible to establish the overlap extension fragment. The product lysGE′eyfp formed in this way was digested with the restriction enzyme BamHI and, after purification of the reaction batch, was employed in ligation reactions with the likewise BamHI-opened and dephosphorylated vector pJC1. The ligation batch was used directly for transformation of E. coli DH5αMCR and the selection of transformants was carried out on LB plates with 50 μg/ml of kanamycin. 20 colonies which grew on these plates and accordingly were kanamycin-resistant were employed for a colony PCR. The colony PCR was carried out in each case with the oligonucleotide combinations described above in order to check whether the fragment lysGE′eyfp was inserted in the vector pJC1. Analysis of the colony PCR in an agarose gel showed the expected PCR product with a size of 1,010 bp in the samples analysed, after which a colony was cultivated for a plasmid preparation on a larger scale. It was possible to demonstrate the presence of the inserted fragment pJC1lysGE′eYFP via the test cleavage with the restriction enzymes BglII, XhoI and PvuI. Sequencing of the insert showed a 100% agreement with the expected sequence.

b) Transformation of Corynebacterium glutamicum with pJC1lysGE′eYFP

Competent cells of the C. glutamicum strains ATCC 13032 and DM1800 were prepared as described by Tauch et al., 2002 (Curr Microbiol. 45(5) (2002), pages 362-7). The strain ATCC 13032 is a wild type which secretes lysine, whereas the strain DM1800 was made into a lysine secretor by gene-directed mutations (Georgi et al. Metab Eng. 7 (2005), pages 291-301) These cells were transformed by electroporation with pJC1lysGE′eYFP as described by Tauch et al. (Curr Microbiol. 45(5) (2002), pages 362-7). The selection of the transformants was carried out on BHIS plates with 25 μg/ml of kanamycin. Colonies which grew on these plates and accordingly were kanamycin-resistant, were checked for the presence of the vectors by plasmid preparations and test cleavages with the enzymes BglII, XhoI and PvuI. In each case one correct clone was designated ATCC 13032 pJC1lysGE′eYFP and DM1800 pJC1lysGE′eYFP.

### c) Detection of the Lysine-Specific Fluorescence

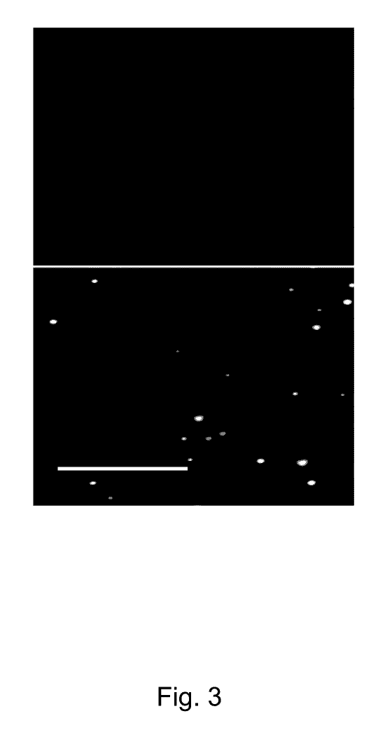

The in vivo emission of fluorescence was tested via confocal microscopy with a Zeiss AxioImager M1. For this purpose, 3 μl of cell suspension of the strains ATCC 13032 pJC1lysGE′eYFP and DM1800 pJC1lysGE′eYFP placed on a slide, to which a thin layer of 1% strength agarose had been applied beforehand for immobilization. The immobilized suspension was excited with light of wavelength 514 nm and an exposure time of 700 ms. The fluorescence emission measurement of eYFP was carried out using a broadband filter in the range of from 505 nm to 550 nm Fluorescent cells were documented digitally with the aid of the AxioVision 4.6 software. It can be seen in the image that emission of fluorescence occurs only in the case of the lysine-forming strain DM1800 pJC1lysGE′Eyfp (FIG. 3 (bottom)), whereas the strain ATCC13032 pJC1lysGE′eYFP (FIG. 3 (top)) which does not form lysine is not fluorescent.

### Example 2

Production of a cell according to the invention according to the second embodiment by the example of a cell in which the expression of an autofluorescent protein is regulated down by the adenine riboswitch (ARS) and in which the expression of the autofluorescent protein depends on the intracellular adenine concentration.

The adenine riboswitch (ARS) from Bacillus subtilis (see Mandai and Breaker, Nat Struct Mol Biol, 11 (2004), pages 29-35) was first amplified, starting from genomic DNA from Bacillus subtilis, with the primers ARS_for (SEQ ID NO:42) and ARS_rev (SEQ ID NO:43). In a second PCR, starting from the ARS amplificate purified by means of the Qiagen MinElute Gel Extraction Kit, using the primers ARS_for_BamHI and ARS_rev_NdeI, an ARS amplificate having a 5′-terminal BamHI and 3′-terminal NdeI cleavage site was amplified and cleaved with these restriction enzymes.

The reporter gene eyfp was amplified on the basis of pEKE×2-EYFP with the primers EYFP_for_NdeI (SEQ ID NO:44) and EYFP_rev_EcoRI (SEQ ID NO:45), restricted with the enzymes NdeI and EcoRI and likewise purified by means of the Qiagen MinElute Gel Extraction Kit.

ARS_for:
(SEQ ID NO: 42)
5′-TCAACTGCTATCCCCCCTGTTA-3′
ARS_rev:
(SEQ ID NO: 43)
5′-AAACTCCTTTACTTAAATGTTTTGATAAATAAA-3′
EYFP_for_NdeI:
(SEQ ID NO: 44)
5′-TACATATGGTGAGCAAGGGCGA-3′
EYFP_rev_EcoRI:
(SEQ ID NO: 45)
5′-TAGAATTCTTATCTAGACTTGTACAGCTCG-3′

The two restricted PCR products were ligated together into the vector pEKEx2, ligated with BamHI and EcoRI beforehand, and were therefore placed under the control of the IPTG-inducible promoter ptac. E. coli XL1 blue was then transformed with the ligation batch.

Kanamycin-resistant transformants were tested by means of colony PCR for the presence of the construct pEKEx2-ARS-EYFP (primers pEKEx2_for (SEQ ID NO:46) and EYFP_rev (SEQ ID NO:47)) and the plasmid was purified for further analysis.

For verification of the construct prepared, pEKEx2-ARS-EYFP, this was cleaved with the restriction enzyme NdeI and tested with the aid of the band pattern.

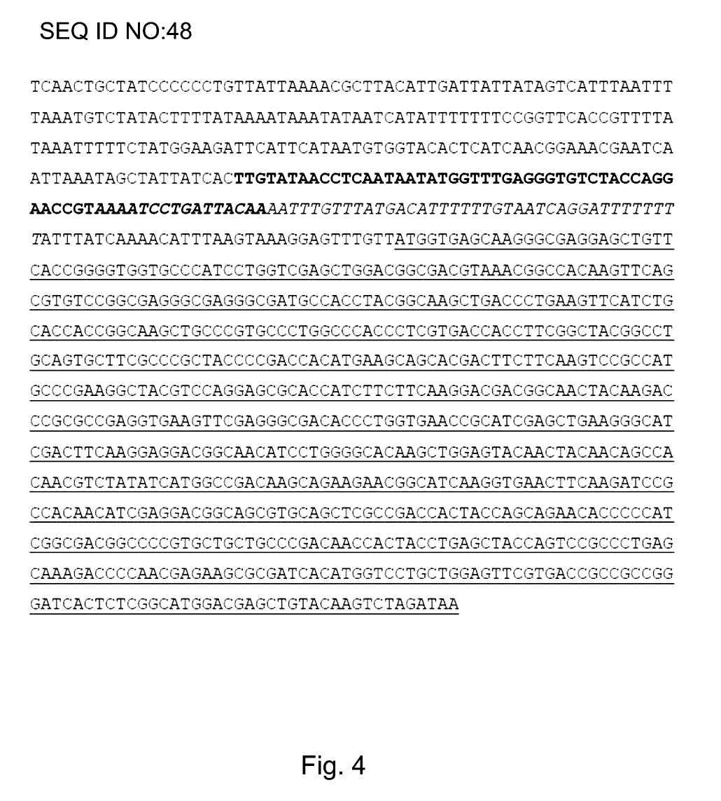

A sequencing (SEQ ID NO:48) of the adenine sensor shown in FIG. 4 confirmed the intact fusion of the adenine-dependent riboswitch (ydhL) with the autofluorescent protein EYFP.

pEKEx2_for:
(SEQ ID NO: 46)
5′-CGGCGTTTCACTTCTGAGTTCGGC-3′
EYFP_rev:
(SEQ ID NO: 47)
5′-TAGAATTCTTATCTAGACTTGTACAGCTCG-3′

### Example 3

Production of a cell according to the invention according to the first embodiment by the example of a cell in which a gene sequence coding for an autofluorescent protein is under the control of the brnFE promoter and in which the expression of the autofluorescent protein depends on the intracellular L-methionine concentration.

a) Construction of the Vector pJC1lrp-brnF′eYF

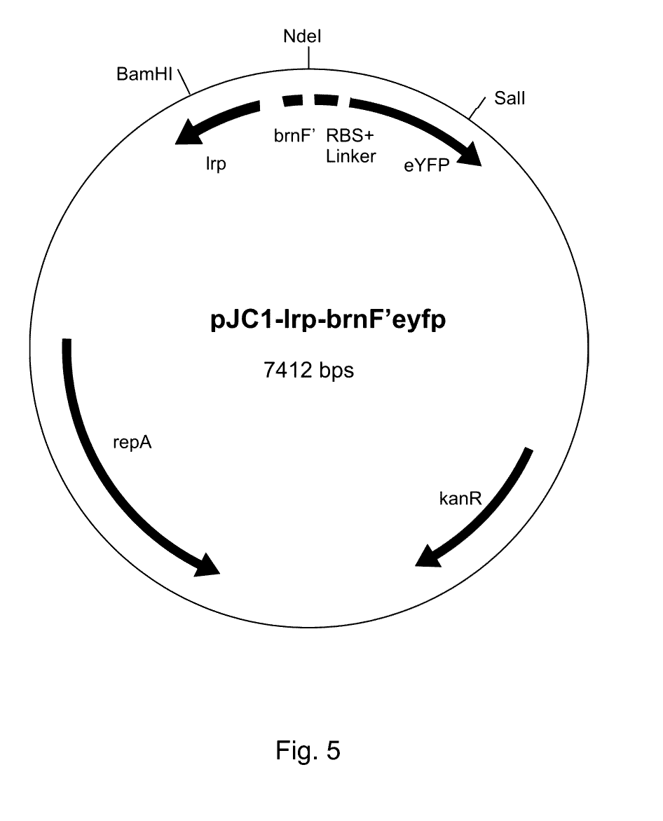

The procedure for the construction of the fusion of brnF with the reporter gene eyfp was as follows. In two separate reactions, first the coding lrp and the first 30 nucleotides of the brnF sequence (brnF′) together with the intergene region (560 bp) were amplified with the oligonucleotide pair lrp-fw-A-BamHI (SEQ ID NO:50)/lrp-brnF-rv-I-NdeI (SEQ ID NO:51) and eyfp (751 bp) was amplified with the oligonucleotide pair eyfp-fw-H-NdeI (SEQ ID NO:52)/eyfp-rv-D-SalI (SEQ ID NO:53). Genomic DNA from C. glutamicum and the vector pEKEx2-yfp-tetR (Frunzke et al., 2008, J. Bacteriol. 190: 5111-5119), which renders possible amplification of eyfp, served as templates. The oligonucleotides fw-A-BamHI and lrp-brnF-rv-I-NdeI were supplemented with 5′-terminal BamHI and NdeI restriction cleavage sites and the oligonucleotides eyfp-fw-H-NdeI and eyfp-rv-D-SalI were supplemented with 5′-terminal NdeI and SalI restriction cleavage sites. After restriction of the lrp-brnF′ amplificates with BamHI and NdeI and of the eyfp amplificate with NdeI and SalI, the lrp-brnF′ amplificates were fused with the eyfp amplificate via the free ends of the NdeI cleavage site in a ligation batch and at the same time cloned into the vector pJC1, which was likewise opened by BamHI and SalI (FIG. 5). The ligation batch was used directly for transformation of E. coli DH5α. The selection of transformants was carried out on LB plates with 50 μg/ml of kanamycin. Colonies which grew on these plates and accordingly were kanamycin-resistant were employed for a colony PCR. In order to check whether the fragment lrp-brnF′eyfp was inserted in the vector pJC1, colony PCR was carried out with oligonucleotides which flank the region of the “multiple cloning site” in the vector pJC1. Analysis of the colony PCR in an agarose gel showed the expected PCR product with a size of 1,530 bp in the samples analysed, after which a colony was cultivated for a plasmid preparation on a larger scale. The presence of the inserted fragment was demonstrated via the test cleavage with the restriction enzymes BamHI, NdeI and SalI. Sequencing of the insert showed a 100% agreement with the expected sequence. The transformation of competent C. glutamicum cells with the vector pJC1lrp-brnF′eYFP was carried out by the method of Tauch and Kirchner (Curr. Microbiol. (2002) 45:362-367), and the strain C. glutamicum ATCC13032 pJC1lrp-brnF′eYFP was obtained.

lrp-fw-A-BamHI
(SEQ ID NO: 50)
5′-GCGCGGATCCTCACACCTGGGGGCGAGCTG-3′
lrp-brnF-rv-I-NdeI
(SEQ ID NO: 51)
5′-GCGCCATATGATATCTCCTTCTTAAAGTTCAGCTTGA
ATGAATCTCTTGCG-3′
eyfp-fw-H-NdeI
(SEQ ID NO: 52)
5′-GCGCCATATGGTGAGCAAGGGCGAGGAG-3′
eyfp-rv-D-SalI
(SEQ ID NO: 53)
5′-GCGCGTCGACTTATCTAGACTTGTACAGCTCGTC-3′
Seq_pJC1_for1
(SEQ ID NO: 54)
5′-CGATCCTGACGCAGATTTTT-3′
Seq_pJC1_rev1
(SEQ ID NO: 55)
5′-CTCACCGGCTCCAGATTTAT-3′

b) Correlation of the Intracellular Methionine Concentration with the Fluorescence Output

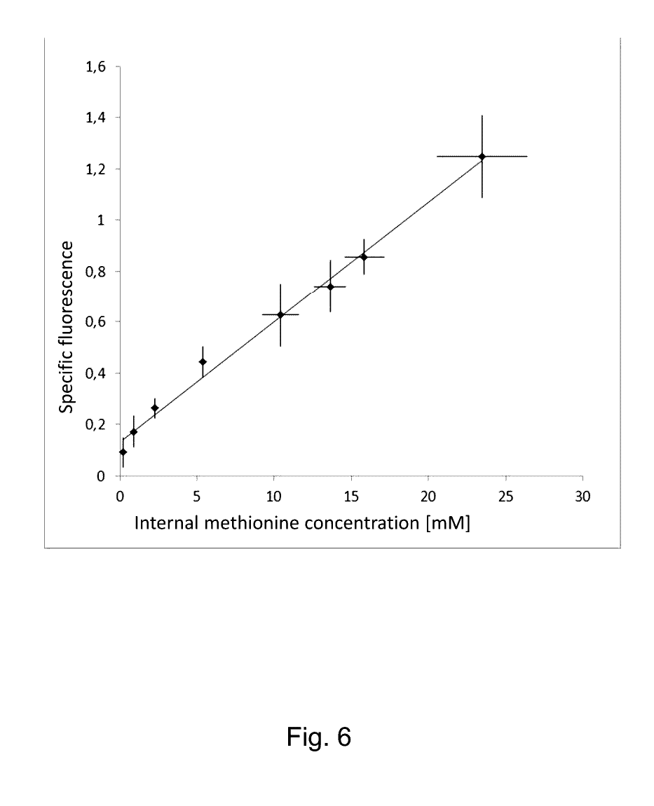

For more detailed characterization, the sensitivity and the dynamic region of the sensor for L-methionine were determined. For this, various internal concentrations of methionine were established with peptides in ATCC13032 pJC1lrp-brnF′eYFP. This method is described, for example, by Trötschel et al., (J. Bacteriol. 2005, 187: 3786-3794). The following dipeptides were employed: L-alanyl-L-methionine (Ala-Met), L-methionyl-L-methionine (Met-Met), and L-alanyl-L-alanine (Ala-Ala). In order to achieve different L-methionine concentrations, the following mixing ratios were used: 0.3 mM Ala-Met plus 2.7 mM Ala-Ala, 0.6 mM Ala-Met plus 2.4 mM Ala-Ala, 0.9 mM Ala-Met plus 2.1 mM Ala-Ala, 1.5 mM Ala-Met plus 1.5 mM Ala-Ala, 2.1 mM Ala-Met plus 0.9 mM Ala-Ala, 2.7 mM Ala-Met plus 0.3 mM Ala-Ala, 3 mM Ala-Met, 3 mM Met-Met, which were added to CGXII medium (Keilhauer et al., 1993, J Bacteriol. 175:5595-603). Cultivation was carried out with 0.6 ml of medium on the microtiter scale (Flowerplate® MTP-48-B) in the BioLector system (m2p-labs GmbH, Forckenbeckstrasse 6, 52074 Aachen, Germany) Seven minutes after addition of the peptides, cells from 200 μl of the cell suspension were separated off from the medium by silicone oil centrifugation and were inactivated as described by Klingenberg and Pfaff (Methods in Enzymology 1967; 10: 680-684). The cytoplasmic fraction of the samples was worked up as described by Ebbinghausen et al. (Arch. Microbiol. (1989), 151:238-244) and the amino acid concentration was quantified by means of reversed phase HPLC as described by Lindroth and Mopper (Anal. Chem. (1979) 51, 1167-1174). The fluorescence of the cultures of ATCC13032 pJC1lrp-brnF′eYFP with the various peptide concentrations was detected online with the BioLector system (m2p-labs GmbH, Forckenbeckstrasse 6, 52074 Aachen, Germany). The correlation of the internal L-methionine concentration with the fluorescence output signal is shown in FIG. 6. It can be seen that the sensor plasmid pJC1lrp-brnF′eYFP renders possible intracellular detection of methionine in a linear range of approx. 0.2-25 mM. An accumulation of methionine can already be detected in the lower mM region (<1 mM).

### Example 4

Use of a metabolite sensor for isolation of cells with increased lysine formation and identification of new mutations which lead to lysine formation.

a) Construction of a Recombinant Wild Type of Corynebacterium glutamicum with the Lysine Sensor pSenLysTK-C

The vector pJC1 is described by Cremer et al. (Molecular and General Genetics, 1990, 220:478-480). This vector was cleaved with BamHI and SalI, and ligated with the 1,765 kb fragment BamHI-<-EYFP-lysE′-lysG->-SalI (SEQ ID No. 56), synthesized by GATC (GATC Biotech AG, Jakob-Stadler-Platz 7, 78467 Konstanz).

The resulting vector pSenLysTK was digested with the restriction enzyme BamHI, and ligated with the 2,506 fragment BamHI-T7terminator-<-crimson—lacIQ->-BamHI (SEQ ID NO:57) synthesized by GATC (GATC Biotech AG, Jakob-Stadler-Platz 7, 78467 Konstanz).

The resulting vector was called pSenLysTK-C. It comprises EYFP as transcriptional fusion and the protein crimson as a live marker. The sensor plasmid pSenLysTK-C was introduced into competent cells of the wild type as described by Tauch et al. (Curr. Microbiol. 45 (2002), pages 362-7), and the strain Corynebacterium glutamicum ATCC13032 pSenLysTK-C was obtained.

b) Mutagenesis of Corynebacterium glutamicum ATCC13032 pSenLysTK-C

The strain ATCC13032 pSenLysTK-C produced was grown overnight in “Difco Brain Heart Infusion” medium (Difco, Becton Dickinson BD, 1 Becton Drive, Franklin Lakes, N.J. USA) at 30° C., and to 5 ml of this culture 0.1 ml of a solution of 0.5 mg of N-methyl-N-nitroso-N′-nitroguanidine, dissolved in 1 ml of dimethylsulfoxide, was added. This culture was shaken at 30° C. for 15 minutes. The cells were then centrifuged off at 4° C. and 2,500 g and resuspended in 5 ml of 0.9% NaCl. The centrifugation step and the resuspension were repeated. 7.5 ml of 80% strength glycerol were added to the cell suspension obtained in this way and aliquots of this mutated cell suspension were stored at −20° C.

### c) High Throughput Cytometry (HT-FACS=“High Throughput Fluorescence Activated Cell Sorting”) and Cell Sorting

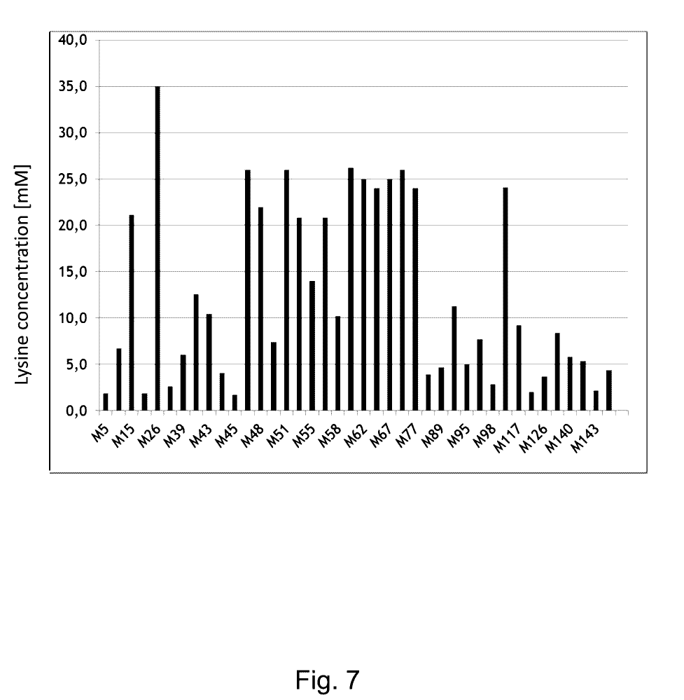

200 μl of the cell suspension obtained under b) were added to 20 ml of CGXII-Kan25 liquid medium (Keilhauer et al., J. Bacteriol. 1993; 175(17):5595-603) and the culture was incubated at 30° C. and 180 rpm. After 45 minutes, isopropyl β-D-thioglactopyrano side was added in a final concentration of 0.1 mM. After further incubation for 2 hours, the analysis of the optical properties and the sorting of cell particles on the FACS Aria II cell sorter from Becton Dickinson (Becton Dickinson BD, 1 Becton Drive, Franklin Lakes, N.J. USA) were carried out. The FACS settings as threshold limits for the “forward scatter” and “side scatter” were 500 at an electronic amplification of 50 mV for the “forward scatter” (ND filter 1.0) and 550 mV for the “side scatter”. Excitation of EYFP was effected at a wavelength of 488 nm and detection by means of “parameter gain” (PMT) of from 530 to 30 at 625 mV. Excitation of crimson was effected at a wavelength of 633 nm and detection by means of PMT of from 660 to 20 at 700 mV. 2 million crimson-positive cells were sorted in 20 ml of CGXII-Kan25 and the culture was cultivated at 180 rpm and 30° C. for 22 hours. Isopropyl β-D-thioglactopyranoside was then added again in a final concentration of 0.1 mM. After a further 2 hours, 18,000,000 cells were analysed for EYFP and crimson fluorescence at an analysis speed of 10,000 particles per second, and 580 cells were sorted out, and were automatically deposited on BHIS-Kan25 plates with the aid of the FACS Aria II cell sorter. The plates were incubated at 30° C. for 16 h. Of the 580 cells deposited, 270 grew. These were all transferred into 0.8 ml of CGXII-Kan25 in microtiter plates and cultivated at 400 rpm and 30° C. for 48 h. The plates were centrifuged in the microtiter plate rotor at 4,000×g for 30 min at 4° C. and the supernatants were diluted 1:100 with water and analysed by means of HPLC. 185 clones were identified as lysine-forming agents. For more detailed characterization, an analysis of 40 of these clones for product formation was again carried out in 50 ml of CGXII-Kan25 in shaking flasks. While the starting strain ATCC13032 pSenLysTK-C secretes no lysine, the 40 mutants form varying amounts of lysine in the range of 2-35 mM (FIG. 7).

d) Identification of Mutations in lysC, Horn, thrB and thrC

For further characterization of the 40 mutants, their chromosomal DNA was isolated by means of the DNeasy kit from Qiagen (Qiagen, Hilden, Germany). The gene lysC was amplified with the primers lysC-32F (SEQ ID NO:58) and lysC-1938R (SEQ ID NO:59) and the amplificates were sequenced by Eurofins MWG Operon (Anzingerstr. 7a, 85560 Ebersberg, Germany).

lysC-32F
(SEQ ID NO: 58)
5′-GAACATCAGCGACAGGACAA-3′
lysC-1938R
(SEQ ID NO: 59)
5′-GGGAAGCAAAGAAACGAACA-3′

The already known mutations T311I, T308I, A279T, A279V and A279T were obtained. In addition, the new mutations H357Y (cac->tac), T313I (acc->atc), G277D (ggc->gac) and G277S (ggc->agc) were obtained. The coding triplet of the wild type, followed by the correspondingly mutated triplet of the mutants, is given in each case in parentheses.

The gene horn was amplified with the primers hom-289F (SEQ ID NO:60) and thrB-2069R (SEQ ID NO:61) and the amplificates were sequenced by Eurofins MWG Operon (Anzingerstr. 7a, 85560 Ebersberg, Germany).

hom-289F
(SEQ ID NO: 60)
5′-CCTCCCCGGGTTGATATTAG-3′
thrB-2069R
(SEQ ID NO: 61)
5′-GGCCAGCACGAATAGCTTTA-3′

The new mutations A346V (gct->gtt), V211F (gtc->ttc), G241S (ggt->agt), A328V (gct->gtt), T233I (acc->atc), and the double mutation R158C (cgc->tgc) T351I (acc->atc) were obtained.

Further sequencing of thrB in the mutants with the primer pair hom-1684F (SEQ ID NO:62) and thrB-2951R (SEQ ID NO:63) gave the new mutation S102F (tcc->ttc).

hom-1684F
(SEQ ID NO: 62)
5′-AGGAATCTCCCTGCGTACAA-3′
thrB-2951R
(SEQ ID NO: 63)
5′-CCGGATTCATCCAAGAAAGC-3′

Further sequencing of thrC in the mutants with the primer pair thrC-22F (SEQ ID NO:64) and thrC-2046R (SEQ ID NO:65) gave the new mutation A372V (gcc->gtc).

thrC-22F
(SEQ ID NO: 64)
5′-GCCTTAAAACGCCACTCAAT-3′
thrC-2046R
(SEQ ID NO: 65)
5′-GGCCGTTGATCATTGTTCTT-3′

e) Identification of a Mutation in murE

For further identification of mutations in mutants which contain mutations neither in lysC, nor horn, thrB or thrC, murE was additionally sequenced. The gene murE was amplified with the primers murE-34F (SEQ ID NO:66) and murE-1944R (SEQ ID NO:67), and the amplificates were sequenced by GATC (GATC Biotech AG, Jakob-Stadler-Platz 7, 78467 Konstanz).

murE-34F
(SEQ ID NO: 66)
5′-AACTCCACGCTGGAGCTCAC-3′
murE-1944R
(SEQ ID NO: 67)
5′-AGAACGCGGAGTCCACG-3′

The murE gene sequence (SEQ ID NO:69), which contains a C to T transition in nucleotide 361 (ctc->ttc), which in the MurE protein (SEQ ID NO:68) leads to the amino acid exchange L121F in position 121 of the protein, was determined.

f) Effect of the murE Mutation on Lysine Formation in the Wild Type

By means of the primers 7-39-L-F (SEQ ID NO:70) and 7-39-R-R (SEQ ID NO:71), 1 kb of the gene murE was amplified with chromosomal DNA of the C. glutamicum mutant M39 from Example e) and a murE fragment which carries the newly identified mutations was thus obtained. The amplificate obtained was cloned via BamHI and SalI into the vector pK19mobsacB which is not replicative in C. glutamicum (Schäfer et al., Gene 1994; 145:69-73) and introduced into the wild-type genome by means of homologous recombination (Tauch et al., Curr. Microbiol. 45 (2002), pages 362-7; Schäfer et al., Gene 1994; 145:69-73). The resulting strain C. glutamicum Lys39 was then cultivated in 50 ml of BHIS-Kan25 at 30° C. and 130 rpm for 12 h. 500 μl of this culture were transferred into 50 ml of CGXII-Kan25 and cultivated again at 30° C. and 130 rpm for 24 h. Starting from this, the 50 ml of CGXII main culture with an initial OD of 0.5 were inoculated and this culture was cultivated at 130 rpm and 30° C. for 48 h. The culture supernatant was diluted 1:100 with water and the L-lysine concentration obtained in Table 1 was determined by means of HPLC.

7-39-L-F
(SEQ ID NO: 70)
5′-TAGGATCCCGACAACATCCCACTGTCTG-3′
7-39-R-R
(SEQ ID NO: 71)
5′-AAGTCGACGTCTGCTTCTTGCCCAAGG-3′

TABLE 1

| Strain | L-Lysine (mM) |
|---|---|
| C. glutamicum ATCC13032 | 0.5 |
| C. glutamicum Lys39 | 3.4 |

L-Lysine in the supernatant of C. glutamicum

SEQUENCES

SEQ ID No: 1

| | |
|---|---|
| agtttgcgca tgagacaaaa tcaccggttt tttgtgttta tgcggaatgt ttatctgccc | 60 |
| cgctcggcaa aggcaatcaa ttgagagaaa aattctcctg ccggaccact aagatgtagg | 120 |
| ggacgctga | 129 |

SEQ ID NO: 2

| | |
|---|---|
| ctattcgcgc aaggtcatgc cattggccgg caacggcaag gctgtcttgt agcgcacctg | 60 |
| tttcaaggca aaactcgagc ggatattcgc cacacccggc aaccgggtca ggtaatcgag | 120 |
| aaaccgctcc agcgcctgga tactcggcag cagtacccgc aacaggtagt ccgggtcgcc | 180 |
| cgtcatcagg tagcactcca tcacctcggg ccgttcggca atttcttcct cgaagcggtg | 240 |
| cagcgactgc tctacctgtt tttccaggct gacatggatg aacacattca catccagccc | 300 |
| caacgcctcg ggcgacaaca aggtcacctg ctggcggatc acccccagtt cttccatggc | 360 |
| ccgcacccgg ttgaaacagg gcgtgggcga caggttgacc gagcgtgcca gctcggcgtt | 420 |
| ggtgatgcgg gcgttttcct gcaggctgtt gagaatgccg atatcggtac gatcgagttt | 480 |
| gcgcat | 486 |

SEQ ID NO: 3

| | |
|---|---|
| aacctatagt gaatgtgtct gaaaataacg acttcttatt gtaagcgtta tcaatacgca | 60 |
| agttgacttg aaaagccgac atgacaatgt ttaaatggaa aagtc | 105 |

SEQ ID NO: 4

| | |
|---|---|
| atggctttat tacaaaaaac aagaattatt aactccatgc tgcaagctgc ggcagggaaa | 60 |
| ccggtaaact tcaaggaaat ggcggagacg ctgcgggatg taattgattc caatattttc | 120 |
| gttgtaagcc gcagagggaa actccttggg tattcaatta accagcaaat tgaaaatgat | 180 |
| cgtatgaaaa aaatgcttga ggatcgtcaa ttccctgaag aatatacgaa aaatctgttt | 240 |
| aatgtccctg aaacatcttc taacttggat attaatagtg aatatactgc tttccctgtt | 300 |
| gagaacagag acctgtttca agctggttta acaacaattg tgccgatcat cggaggcggg | 360 |
| gaaagattag gaacacttat tctttcgcgt ttacaagatc aattcaatga cgatgactta | 420 |
| attctagctg aatacggcgc aacagttgtc ggaatggaaa tcctaagaga aaaagcagaa | 480 |
| gaaattgaag aggaagcaag aagcaaagct gtcgtacaaa tggctatcag ctcgctttct | 540 |
| tacagtgagc ttgaagcaat tgagcacatt tttgaggagc ttgacggaaa tgaaggtctt | 600 |
| cttgttgcaa gtaaaattgc tgaccgtgtc ggcattaccc gttctgttat tgtgaacgca | 660 |
| ctcagaaagc tggagagcgc cggtgttatc gagtctagat cattaggaat gaaaggtact | 720 |
| tatatcaagg tactaaacaa caaattccta attgaattag aaaatctaaa atctcattaa | 780 |

SEQ ID NO: 5

| | |
|---|---|
| tgttgttttt atgtcagtga gcggcgcttt tcgtaggcgt atttggaaaa atttaagccg | 60 |
| gtccgtggaa taagcttata acaaaccaca agaggcggtt gccatg | 106 |

SEQ ID NO: 6

| | |
|---|---|
| tcaaatatgc ttctgtgcca ccggaatcac ccgcttctcc ttcaccgcct tgaacgagaa | 60 |
| gctcgaatag atctccttca cccccggcag ccgctgcagt acctcgcggg tgaactcgcc | 120 |
| gaacgactcc agatcccgcg ccagaatctc cagcaggaag tcatagcgcc cggagatgtt | 180 |
| gtggcacgcc acgatttcgg ggatatccat cagccgctgc tcgaatgccc gggccatctc | 240 |
| cttgctgtgc gaatccatca tgatgctgac gaaggcggtc actccgaagc ccagtgcctt | 300 |
| gggtgacagg atggcctgat agccggtgat gtagcccgac tcctccagca gcttgacccg | 360 |
| ccgccagcac ggcgaggtgg tcagggcgac gctgtcggcg agctcggcca cggtcagtcg | 420 |
| ggcattgtct tgcagcgcgg ccagcagtgc gcggtcggta cggtcgatgg cgctaggcat | 480 |

SEQ ID NO: 7

| | |
|---|---|
| tttttagacc ttgcgcgatt tcgtagcgcc gataaccttt atcatctggt tccagggctg | 60 |
| ccttggatgg cgacacctcc aggcttgaat gaatctcttg cgttttttgc acactacaat | 120 |
| catcacacaa ttgccgggta gttttgttgc cagtttgcgc acctcaacta ggctattgtg | 180 |
| caatat | 186 |

SEQ ID NO: 8

| | |
|---|---|
| atgaagctag attccattga tcgcgcaatt attgcggagc ttagcgcgaa tgcgcgcatc | 60 |
| tcaaatctcg cactggctga caaggtgcat ctcactccgg gaccttgctt gaggagggtg | 120 |
| cagcgtttgg aagccgaagg aatcattttg ggctacagcg cggacattca ccctgcggtg | 180 |
| atgaatcgtg gatttgaggt gaccgtggat gtcactctca gcaacttcga ccgctccact | 240 |
| gtagacaatt ttgaaagctc cgttgcgcag catgatgaag tactggagtt gcacaggctt | 300 |
| tttggttcgc cagattattt tgtccgcatc ggcgttgctg atttggaggc gtatgagcaa | 360 |
| tttttatcca gtcacattca aaccgtgcca ggaattgcaa agatctcatc acgttttgct | 420 |
| atgaaagtgg tgaaaccagc tcgcccccag gtgtga | 456 |

SEQ ID NO: 9

| | |
|---|---|
| aacttattcc cttttcaact tccaaatcac caaacggtat ataaaaccgt tactcctttc | 60 |
| acgtccgtta taaatatgat ggctattag | 89 |

SEQ ID NO: 10

| | |
|---|---|
| atgaaattac aacaacttcg ctatattgtt gaggtggtca atcataacct gaatgtctca | 60 |
| tcaacagcgg aaggacttta cacatcacaa cccgggatca gtaaacaagt cagaatgctg | 120 |
| gaagacgagc taggcattca aattttttcc cgaagcggca agcacctgac gcaggtaacg | 180 |
| ccagcagggc aagaaataat tcgtatcgct cgcgaagtcc tgtcgaaagt cgatgccata | 240 |
| aaatcggttg ccggagagca cacctggccg gataaaggtt cactgtatat cgccaccacg | 300 |
| catacccagg cacgctacgc attaccaaac gtcatcaaag gctttattga gcgttatcct | 360 |
| cgcgtttctt tgcatatgca ccagggctcg ccgacacaaa ttgctgatgc cgtctctaaa | 420 |
| ggcaatgctg atttcgctat cgccacagaa gcgctgcatc tgtatgaaga tttagtgatg | 480 |
| ttaccgtgct accactggaa tcgggctatt gtagtcactc cggatcaccc gctggcaggc | 540 |
| aaaaaagcca ttaccattga agaactggcg caatatccgt tggtgacata taccttcggc | 600 |
| tttaccggac gttcagaact ggatactgcc tttaatcgcg cagggttaac gccgcgtatc | 660 |
| gttttcacgg caacggatgc tgacgtcatt aaaacttacg tccggttagg gctgggggta | 720 |
| ggggtcattg ccagcatggc ggtggatccg gtcgccgatc ccgaccttgt gcgtgttgat | 780 |
| gctcacgata tcttcagcca cagtacaacc aaaattggtt ttcgccgtag tactttcttg | 840 |
| cgcagttata tgtatgattt cattcagcgt tttgcaccgc atttaacgcg tgatgtcgtt | 900 |
| gatgcggctg tcgcattgcg ctctaatgaa gaaattgagg tcatgtttaa agatataaaa | 960 |
| ctgccggaaa aataa | 975 |

SEQ ID NO: 11

| | |
|---|---|
| tttttattac ataaatttaa ccagagaatg tcacgcaatc cattgtaaac attaaatgtt | 60 |
| tatcttttca tgatatcaac ttgcgatcct gatgtgttaa taaaaaacct caagttctca | 120 |
| cttacagaaa cttttgtgtt atttcaccta atctttagga ttaatccttt tttcgtgagt | 180 |
| aatcttatcg ccagtttggt ctggtcagga aatagttata catcatgacc cggactccaa | 240 |
| attcaaaaat gaaattagga gaagagcatg | 270 |

SEQ ID NO: 12

| | |
|---|---|
| ttattctgaa gcaagaaatt tgtcgagata aggtacaaca taaggaacag aagtctggaa | 60 |
| tataccattt tcaatccagt aaagggtgtt tgcccctggg cgtaaattaa aggcggtgag | 120 |
| atatgcatca gctgcttccc ggttcatccc cttcatttca taaaccttgc caagcaacac | 180 |
| ataatttagc caggacattt caagatcaat gccagtattt atcgcctggt aagactcatc | 240 |
| tgttttacct tttaccagag cactgaccgc ttttatttga tatataatgg acaggttgtt | 300 |
| caattccggc agtgtaacaa tgttatctat ttctgtgttc agtgctgcta attgtttttc | 360 |
| atctaaagga tgttgagaat ggcgcacgat atcaactaat gctttttctg ctctcgcgta | 420 |
| ggtaaattct ggggatgatt gaacaatctc acctaataat tcactggcac ggttcaatga | 480 |
| tttatcatcg ccatgcagta aataatcatg tgcctgataa aaattagtta ataacgcacc | 540 |
| acgatgcggc aaaattttct ggagcgtctc ctgcattcgt tgtggccacg gttggtttaa | 600 |
| cgcttttgat aaactctcca gtaaatcatt ttgaatcgcc agctgattac cgttagtgat | 660 |
| gacataacgt ttatccagca tggttgaacc atctgcattg tctaccaatt ttatcgacat | 720 |
| aaagcattgt tgagcacggt attggcgctg attaacaaac gcaatagata atgttttacc | 780 |
| ggaactgctc ggttcatcaa tgttgtagtt gattttgtca tgcaccataa aggtggagaa | 840 |
| ggtgttaagt gatgtcgcca ccaaatcacc cacgcctatc gcgtaagaga gctgatacgg | 900 |
| ggaactccag ctgttacaac ttttatttac catattaatg tcaatatcgc gtggattgag | 960 |
| caaaatacgc gatttgctca taggaagacg tgtatcaaga cttgaaaacg ctaccagtgc | 1020 |
| tacacagata cctaacgaca acaggaaaaa aaaccatacc caaaaggtag tgaatcgttt | 1080 |
| gcttttaact ggggattgtt caggtggcgt tgcggtgttt tgaatgttaa gactgtggga | 1140 |
| gggagaatct gtggcaggaa ccgcctctgg tataggggga ggcgaagata gcattatttc | 1200 |
| ctctccctct tcttcgctgt accagataac cggcaccatt aatttatagc cgcgctttgg | 1260 |
| tacagtagcg atatagacag gactatcttc atcattatct tttaatgact tacgtagttc | 1320 |
| tgagatactc tgcgtcacaa cgtgattggt gacaatactt ctcttccaga cattatcgat | 1380 |
| aagttcatcc ctgctaagta cttcgccact gtgttgagca aagaaaacca gaagatcgat | 1440 |
| taatctcggc tcaagggtaa gttgacgccc attgcggcta atttggttta tggacggagt | 1500 |
| aacaagccat tcgccaacgc gaactacagg ttgttgcat | 1539 |

SEQ ID NO: 13

| | |
|---|---|
| tagaccaaga tgttca | 16 |

SEQ ID NO: 14

| | |
|---|---|
| ctaaattgag tagtccgcag gtggagccga caacaactgc cgagccaaat cgcgagccgt | 60 |
| ctcaagagga ctgatgttgt ggaccaatcg agatccagca agtccaccat caaggaacac | 120 |
| caacagctga ttcgcctggg tggtgcctgg gtagccgttc ttctcagtga gcaaatcagt | 180 |
| cagagtctta tgacaccact cgcggtgctc taacactgct gcaacaatgc ccttttcgct | 240 |
| atcagtttcg gggcgagggt actcactagc cgcattctga aagtgcgagc cgcggaaatc | 300 |
| tttttctggt tcttcctcaa tgcactgatc aaagaacgcg atgattttat cttccggatc | 360 |
| cttcataccg acggtgcgct cacgccacgc ttcacgccac agctgatcga ggttctccag | 420 |
| gtatgcaata accaaggcgt ccttcgatcc gaaaagggaa tagaggctcg ccttcgccac | 480 |
| gtcagcttca cggaggatac gatcaatacc gatgacgcga ataccttctg tggtgaaaag | 540 |
| gttggttgcg ctatcgagga gacgctgtcg ggggcttggt cgattgcgac gacggtttgc | 600 |
| cccggcactt gttttactct tgcctgaagc gctagcagcc ac | 642 |

SEQ ID NO: 15

| | |
|---|---|
| cttattagtt tttctgattg ccaattaata ttatcaattt ccgctaataa caatcccgcg | 60 |
| atatagtctctgcatcagatacttaattcg gaatatccaac | 101 |

SEQ ID NO: 16

| | |
|---|---|
| atgaaacgcc cggactacag aacattacag gcactggatg cggtgatacg tgaacgagga | 60 |
| tttgagcgcg cggcacaaaa gctgtgcatt acacaatcag ccgtctcaca gcgcattaag | 120 |
| caactggaaa atatgttcgg gcagccgctg ttggtgcgta ccgtaccgcc gcgcccgacg | 180 |
| gaacaagggc aaaaactgct ggcactgctg cgccaggtgg agttgctgga agaagagtgg | 240 |
| ctgggcgatg aacaaaccgg ttcgactccg ctgctgcttt cactggcggt caacgccgac | 300 |
| agtctggcga cgtggttgct tcctgcactg gctcctgtgt tggctgattc gcctatccgc | 360 |
| ctcaacttgc aggtagaaga tgaaacccgc actcaggaac gtctgcgccg cggcgaagtg | 420 |
| gtcggcgcgg tgagtattca acatcaggcg ctgccgagtt gtcttgtcga taaacttggt | 480 |
| gcgctcgact atctgttcgt cagctcaaaa ccctttgccg aaaaatattt ccctaacggc | 540 |
| gtaacgcgtt cggcattact gaaagcgcca gtggtcgcgt ttgaccatct tgacgatatg | 600 |
| caccaggcct ttttgcagca aaacttcgat ctgcctccag gcagcgtgcc ctgccatatc | 660 |
| gttaattctt cagaagcgtt cgtacaactt gctcgccagg gcaccacctg ctgtatgatc | 720 |
| ccgcacctgc aaatcgagaa agagctggcc agcggtgaac tgattgactt aacgcctggg | 780 |
| ctatttcaac gacggatgct ctactggcac cgctttgctc ctgaaagccg catgatgcgt | 840 |
| aaagtcactg atgcgttact cgattatggt cacaaagtcc ttcgtcagga ttaa | 894 |

SEQ ID NO: 17

| | |
|---|---|
| gcaaagtgtc cagttgaatg gggttcatga agctatatta aaccatgtta agaaccaatc | 60 |
| attttactta agtacttcca taggtcacga tggtgatcat ggaaatcttc | 110 |

SEQ ID NO: 18

| | |
|---|---|
| atgaacccca ttcaactgga cactttgctc tcaatcattg atgaaggcag cttcgaaggc | 60 |
| gcctccttag ccctttccat ttccccctcg gcggtgagtc agcgcgttaa agctctcgag | 120 |
| catcacgtgg gtcgagtgtt ggtatcgcgc acccaaccgg ccaaagcaac cgaagcgggt | 180 |
| gaagtccttg tgcaagcagc gcggaaaatg gtgttgctgc aagcagaaac taaagcgcaa | 240 |
| ctatctggac gccttgctga aatcccgtta accatcgcca tcaacgcaga ttcgctatcc | 300 |
| acatggtttc ctcccgtgtt caacgaggta gcttcttggg gtggagcaac gctcacgctg | 360 |
| cgcttggaag atgaagcgca cacattatcc ttgctgcggc gtggagatgt tttaggagcg | 420 |
| gtaacccgtg aagctaatcc cgtggcggga tgtgaagtag tagaacttgg aaccatgcgc | 480 |
| cacttggcca ttgcaacccc ctcattgcgg gatgcctaca tggttgatgg gaaactagat | 540 |
| tgggctgcga tgcccgtctt acgcttcggt cccaaagatg tgcttcaaga ccgtgacctg | 600 |
| gacgggcgcg tcgatggtcc tgtggggcgc aggcgcgtat ccattgtccc gtcggcggaa | 660 |
| ggttttggtg aggcaattcg ccgaggcctt ggttggggac ttcttcccga aacccaagct | 720 |
| gctcccatgc taaaagcagg agaagtgatc ctcctcgatg agatacccat tgacacaccg | 780 |
| atgtattggc aacgatggcg cctggaatct agatctctag ctagactcac agacgccgtc | 840 |
| gttgatgcag caatcgaggg attgcggcct tag | 873 |

SEQ ID NO: 19

| | |
|---|---|
| gtaccggata ccgccaaaag cgagaagtac gggcaggtgc tatgaccagg actttttgac | 60 |
| ctgaagtgcg gataaaaaca gcaacaatgt gagctttgtt gtaattatat tgtaaacata | 120 |
| ttgctaaatg tttttacatc cactacaacc atatcatcac aagtggtcag acctcctaca | 180 |
| agtaaggggc ttttcgtt | 198 |

SEQ ID NO: 20

| | |
|---|---|
| atggtcatta aggcgcaaag cccggcgggt ttcgcggaag agtacattat tgaaagtatc | 60 |
| tggaataacc gcttccctcc cgggactatt ttgcccgcag aacgtgaact ttcagaatta | 120 |
| attggcgtaa cgcgtactac gttacgtgaa gtgttacagc gtctggcacg agatggctgg | 180 |
| ttgaccattc aacatggcaa gccgacgaag gtgaataatt tctgggaaac ttccggttta | 240 |
| aatatccttg aaacactggc gcgactggat cacgaaagtg tgccgcagct tattgataat | 300 |
| ttgctgtcgg tgcgtaccaa tatttccact atttttattc gcaccgcgtt tcgtcagcat | 360 |
| cccgataaag cgcaggaagt gctggctacc gctaatgaag tggccgatca cgccgatgcc | 420 |
| tttgccgagc tggattacaa catattccgc ggcctggcgt ttgcttccgg caacccgatt | 480 |
| tacggtctga ttcttaacgg gatgaaaggg ctgtatacgc gtattggtcg tcactatttc | 540 |
| gccaatccgg aagcgcgcag tctggcgctg ggcttctacc acaaactgtc ggcgttgtgc | 600 |
| agtgaaggcg cgcacgatca ggtgtacgaa acagtgcgtc gctatgggca tgagagtggc | 660 |
| gagatttggc accggatgca gaaaaatctg ccgggtgatt tagccattca ggggcgataa | 720 |

SEQ ID NO: 21

| | |
|---|---|
| ttaatttgca tagtggcaat tttttgccag actgaagagg tcataccagt tatgacctct | 60 |
| gtacttataa caacaacgta aggttattgc gctatgcaaa cacaaatcaa agttcgtgga | 120 |
| tatcatctcg acgtttacca gcacgtcaac aacgcccgct accttgaat | 169 |

SEQ ID NO: 22

| | |
|---|---|
| atgggcgtaa gagcgcaaca aaaagaaaaa acccgccgtt cgctggtgga agccgcattt | 60 |
| agccaattaa gtgctgaacg cagcttcgcc agcctgagtt tgcgtgaagt ggcgcgtgaa | 120 |
| gcgggcattg ctcccacctc tttttatcgg catttccgcg acgtagacga actgggtctg | 180 |
| accatggttg atgagagcgg tttaatgcta cgccaactca tgcgccaggc gcgtcagcgt | 240 |
| atcgccaaag gcgggagtgt gatccgcacc tcggtctcca catttatgga gttcatcggt | 300 |
| aataatccta acgccttccg gttattattg cgggaacgct ccggcacctc cgctgcgttt | 360 |
| cgtgccgccg ttgcgcgtga aattcagcac ttcattgcgg aacttgcgga ctatctggaa | 420 |
| ctcgaaaacc atatgccgcg tgcgtttact gaagcgcaag ccgaagcaat ggtgacaatt | 480 |
| gtcttcagtg cgggtgccga ggcgttggac gtcggcgtcg aacaacgtcg gcaattagaa | 540 |
| gagcgactgg tactgcaact gcgaatgatt tcgaaagggg cttattactg gtatcgccgt | 600 |
| gaacaagaga aaaccgcaat tattccggga aatgtgaagg acgagtaa | 648 |

SEQ ID NO: 23

| | |
|---|---|
| ccgtcatact ggcctcctga tgtcgtcaac acggcgaaat agtaatcacg acgtcaggtt | 60 |
| cttaccttaa attttcgacg gaaaaccacg taaaaaacgt cgatttttca agatacaagc | 120 |
| gtgaattttc aggaaatggc ggtgagcatc ac | 152 |

SEQ ID NO: 24

| | |
|---|---|
| atcaccacaa ttcagcaaat tgtgaacatc atcacgttca tctttccctg gttcccaatg | 60 |
| gcccattttc ctgtagtaac gagaacgtcg cgaattcagg cgctctttag actggtcgta | 120 |
| atgaaattca gcaggatcac attatgacc | 149 |

SEQ ID NO: 25

| | |
|---|---|
| gtggcgcatc agttaaaact tctcaaagat gatttttttg ccagcgacca gcaggcagtc | 60 |
| gctgtggctg accgttatcc gcaagatgtc tttgctgaac atacacatga tttttgtgag | 120 |
| ctggtgattg tctggcgcgg taatggcctg catctggttt tgcagaatat tatttattgc | 180 |
| ccggagcgtc tgaagctgaa tcttgactgg cagggggcga ttccgggatt taacgccagc | 240 |
| gcagggcaac cacactggcg cttaggtagc atggggatgg cgcaggcgcg gcaggttatc | 300 |
| ggtcagcttg agcatgaaag tagtcagcat gtgccgtttg ctaacgaaat ggctgagttg | 360 |
| ctgttcgggc agttggtgat gttgctgaat cgccatcgtt acaccagtga ttcgttgccg | 420 |
| ccaacatcca gcgaaacgtt gctggataag ctgattaccc ggctggcggc tagcctgaaa | 480 |
| agtccctttg cgctggataa attttgtgat gaggcatcgt gcagtgagcg cgttttgcgt | 540 |
| cagcaatttc gccagcagac tggaatgacc atcaatcaat atctgcgaca ggtcagagtg | 600 |
| tgtcatgcgc aatatcttct ccagcatagc cgcctgttaa tcagtgatat ttcgaccgaa | 660 |
| tgtggctttg aagatagtaa ctatttttcg gtggtgttta cccgggaaac cgggatgacg | 720 |
| cccagccagt ggcgtcatct caattcgcag aaagattaa | 759 |

SEQ ID NO: 26

| | |
|---|---|
| gtggcgcatc agttaaaact tctcaaagat gatttttttg ccagcgacca gcaggcagtc | 60 |
| gctgtggctg accgttatcc gcaagatgtc tttgctgaac atacacatga tttttgtgag | 120 |
| ctggtgattg tctggcgcgg taatggcctg catgtactca acgatcgccc ttatcgcatt | 180 |
| acccgtggcg atctctttta cattcatgct gacgataaac actcctacgc ttccgttaac | 240 |
| gatctggttt tgcagaatat tatttattgc ccggagcgtc tgaagctgaa tcttgactgg | 300 |
| cagggggcga ttccgggatt taacgccagc gcagggcaac cacactggcg cttaggtagc | 360 |
| atggggatgg cgcaggcgcg gcaggttatc ggtcagcttg agcatgaaag tagtcagcat | 420 |
| gtgccgtttg ctaacgaaat ggctgagttg ctgttcgggc agttggtgat gttgctgaat | 480 |
| cgccatcgtt acaccagtga ttcgttgccg ccaacatcca gcgaaacgtt gctggataag | 540 |
| ctgattaccc ggctggcggc tagcctgaaa agtccctttg cgctggataa attttgtgat | 600 |
| gaggcatcgt gcagtgagcg cgttttgcgt cagcaatttc gccagcagac tggaatgacc | 660 |
| atcaatcaat atctgcgaca ggtcagagtg tgtcatgcgc aatatcttct ccagcatagc | 720 |
| cgcctgttaa tcagtgatat ttcgaccgaa tgtggctttg aagatagtaa ctatttttcg | 780 |
| gtggtgttta cccgggaaac cgggatgacg cccagccagt ggcgtcatct caattcgcag | 840 |
| aaagattaa | 849 |

SEQ ID NO: 27

| | |
|---|---|
| tatcggaaaa aatctgtaac atgagataca caatagcatt tatatttgct ttagtatctc | 60 |
| tctcttgggt gggattc | 77 |

SEQ ID NO: 28

| | |
|---|---|
| gtaattgtgg ctagagtaac aaagactaca aaaccttggg catgggcttg ttactttgaa | 60 |
| attcatcgac gctaag | 76 |

SEQ ID NO: 29

| | |
|---|---|
| cctcgcccct catttgtaca gtctgttacc tttacctgaa acagatgaat gtagaattta | 60 |
| taaaactagc atttgat | 77 |

SEQ ID NO: 30

| | |
|---|---|
| atgatcgtga cacaagataa ggccctagca aatgtttttc gtcagatggc aaccggagct | 60 |
| tttcctcctg ttgtcgaaac gtttgaacgc aataaaacga tcttttttcc tggcgatcct | 120 |
| gccgaacgag tctactttct tttgaaaggg gctgtgaaac tttccagggt gtacgaggca | 180 |
| ggagaagaga ttacagtagc actactacgg gaaaatagcg tttttggtgt cctgtctttg | 240 |
| ttgacaggaa acaagtcgga taggttttac catgcggtgg catttactcc agtagaattg | 300 |
| ctttctgcac caattgaaca agtggagcaa gcactgaagg aaaatcctga attatcgatg | 360 |
| ttgatgctgc ggggtctgtc ttcgcggatt ctacaaacag agatgatgat tgaaacctta | 420 |
| gcgcaccgag atatgggttc gagattggtg agttttctgt taattctctg tcgtgatttt | 480 |
| ggtgttcctt gtgcagatgg aatcacaatt gatttaaagt tatctcatca ggcgatcgcc | 540 |
| gaagcaattg gctctactcg cgttactgtt actaggctac taggggattt gcgggagaaa | 600 |
| aagatgattt ccatccacaa aaagaagatt actgtgcata aacctgtgac tctcagcaga | 660 |
| cagttcactt aa | 672 |

SEQ ID NO: 31

| | |
|---|---|
| atgaccaacg cgcgattgcg agctctggtc gaactggcgg ataccggttc ggtgcgcgcc | 60 |
| gctgctgagc gactcgtggt caccgaatct tcgatctcct cggctttacg cgcattgagc | 120 |
| aacgacatcg gcatcagctt ggtcgaccgg catggccgcg gggtgcggct gactcctgcc | 180 |
| ggcctgcgtt acgtcgaata cgcgcggcgg atcctcggct tgcacgacga ggcgatattg | 240 |
| gctgcccgcg gagaggccga cccggagaat ggctcgatcc ggctggctgc ggtcacctcc | 300 |
| gcgggggaac tgctcatccc cgccgcgttg gcatcgttcc gtgccgcgta ccccggtgtc | 360 |
| gttctgcatc tggaggtggc ggcgcgcagc ttggtgtggc ctatgctggc ccgccacgag | 420 |
| gtcgacctcg ttgtggcggg acggccgccg gacgaattgg tccggaaagt gtgggtgcgc | 480 |
| gccgtcagcc cgaacgcgct tgtcgtcgtg ggaccacccg cggtagcgaa gggattccag | 540 |
| cccgccaccg cgacctggct gctgcgtgag accggatccg gtacccgctc tacgttgacg | 600 |
| gcactgcttg acgacctcga tgtcgcgcca cctcaattgg tgctcggatc gcacggcgcg | 660 |
| gtggttgccg cggcggtggc cgggctgggc gtgacgttgg tgtcgcgtca ggctgtgcag | 720 |
| cgcgaactgg ccgccggcgc actcgtcgaa ctgccggtgc ccggtactcc gataagccgg | 780 |
| ccatggcatg tggtcagcca gatcagtccg acgatgtcga ccgaactgct catcaagcac | 840 |
| ctcttgtccc agcgagacct gggctggcgc gatatcaaca ccacccttcg gggagccgtt | 900 |
| accgcctga | 909 |

SEQ ID NO: 32

| | |
|---|---|
| gtgctggtcc cgcaccgggc ggtggacagc ttccggcggc agctgaccgg ccgctacttc | 60 |
| ggcggcccgg acacctcccg cgagggcgtg ctcttcctgg ccaactacgt cttcgacttc | 120 |

SEQ ID NO: 33

| | |
|---|---|
| atggacgcag acgactgttg ggcgcgggcg ggcaccgtgc ggatccgcct gctcggcccg | 60 |
| gtggagctgg cctgcggcac gcggccggtg ccggtgaccg ggcggcgcca gttgagggtg | 120 |
| gtggccgcgc tcgcgctgga ggccggacgg gtgctctcca ccgcggggct gatcgcctcg | 180 |
| ttgtgggcgg acgagccgcc gcgcaccgcc gcccggcagc tccagaccag cgtgtggatg | 240 |
| atccgccggg cgctcgcctc ggtgggcgcg ccgcagtgcg tcgtccgctc caccccggcc | 300 |
| ggctacctgc tcgacccggc ccactacgaa ctcgacagcg accggttccg gcacgcggtg | 360 |
| ctgaccgccc gggagttgca gcgggacggg cggctggccc aggcccgggc ccgggtcgac | 420 |
| gaggggctgg cgctgtggcg cggccccgcc ctcggcgcgg cggcgggcgc cggactccag | 480 |
| ccccgggccc gccggctgga ggaggaacgg gtcttcgccc tggagcagcg cgccgggctc | 540 |
| gacctcgcgc tcggccgcca cgagacggcc atcggcgaac tcctcgacct catcgcccag | 600 |
| catccgctgc gcgaggcggc ctacgccgac ctgatgctcg ccctgtaccg ttccggccgc | 660 |
| cagtccgacg cgctcgccgt ctaccgcagg gcgcagcggg tgctcgccga cgagctggcc | 720 |
| gtccgccccg gcccccgcct cgccggcctg gagcgggcca tcctgcggca ggacgagtcg | 780 |
| ctgctggccg gcgcggcggt gccctga | 807 |

SEQ ID NO: 34

| | |
|---|---|
| tcaggggcct gcctccagca cgtcggctgc ccggaccagt acggccgagc gggtgccgat | 60 |
| cttcagccgc tccagggcct ttacgggagc caccgggatc ttacggctgc ggtcggtgac | 120 |

SEQ ID NO: 35

| | |
|---|---|
| ctaggaaccc gcggacgtat cgggtggatg gtcggatccc tctgcatcgc cgatgtgtcc | 60 |
| gggaagcccg tgggcgaagg caaccagtcc ggcctgaaga cgggattcga ccccgagctt | 120 |
| cgccagtatc tgggccatat gagccttgac ggtgcgctcg gtgaccccga gcagcgcggc | 180 |
| gatctcacgg ttggagtagc cgtggctcag caggaggaag acctggagct cgcggtcgga | 240 |
| gagtaaatgt acctggctga gcccttccag ccaggggaac tggtcctcgt ggagaaatcg | 300 |
| atcgtcgcca gaatcactgg aatcgcagcc ggaatatggc aaagtctggc ccccgtatga | 360 |
| gcgtgtggtc cttgcatgcc ctaagaggtc atccgacgca tcgagtatca aggcgccgaa | 420 |
| gggcgccacc actgaactat gaagacgtga gggcgatacc acccatgcga cgaatgggtc | 480 |
| ctggacatta ctcatcttga tcatcttatc gcatctacgg ccgggttggg gcgccttggt | 540 |
| gccgcctgct gtcgtgagca gggcccgccg aggcgtgggc aaggcggata aggcggcccg | 600 |
| tgcccggtgt gtgcacggca a | 621 |

SEQ ID NO: 36

| | |
|---|---|
| catcacgaac ctccagccgt gggatcgccc tccggcagca tttatagacg gtttgcttat | 60 |
| cgatccgttt tcacattcac ccgcagtgat aaggaattga taaacgattt tcctagcctg | 120 |
| agcggactat | 130 |

SEQ ID NO: 37

| | |
|---|---|
| gtgcgcgcgg gcgggcgccg ggtccaggtc ggcgggccgc gccagcggac ggtgctggcg | 60 |
| acgctgctgc tcaacgccga ccgcgtggtg tcggtggacg cgctggccga gacggtctgg | 120 |
| ggcgcccggc ccccgtcgac cagccggacg caggtggcga tctgcgtgtc cgcgctgcgc | 180 |
| aaggcgttcc gcgcgagcgg cgccgacgag gtgatcgaga ccgtcgcgcc ggggtacgtc | 240 |
| ctgcgctccg gcgggcaccg gctggacacc ctggacttcg acgaactggt ggcgctggcg | 300 |
| agggcggcgg cccggcaggg ccggggcgcg gaggccgtcc ggctgtacgg ctcggcgctc | 360 |
| gcgctgcgcc ggggcccggt gctggcgaac gtgaccggga cggtgcccga gcacctgtcc | 420 |
| tgccagtggg aggagaccct gctcaccgcc tacgaggagc aggtcgagct gcgcctggcg | 480 |
| ctgggcgagc accgcctgct ggtcgccggg ctcgcggcgg cggtcgagcg gcacccgctg | 540 |
| cgcgaccggc tctacggcct gctcatcatc gcccagtacc gctccggcca ccgggccgcg | 600 |
| gcgctggaga cgttcgcccg gttgcgccgc cgctcggtcg acgagctcgg cctggagccg | 660 |
| gggatggagc tgcgccggct gcacgagcgc atcctgcgcg acgaggaccg cccggcggtc | 720 |
| gagcgcccgc cgtcgcagct gcccgccgcg acgcaggtgt tcgtcgggcg cgccgaggag | 780 |
| ctggcggtgc tggaccggct ggccgccgag gacgggcagg cgggcgcgcc gccgctcgga | 840 |
| ctgctggtcg gcggcgtcgg cgtgggcaag accgcgctgg cggtgcggtg ggcgcacgcc | 900 |
| aacgccgacc tgttccccga cggccagctg ttcgtcgacc tgggcgggca cgacccgcac | 960 |
| cacccgccgt cggcccccgg cgccgtgctc gcgcacctgc tgcacgcgct gggcgtgccg | 1020 |
| cccgagcggg tgccggtcgc cgccgaacga cccgcgctgt tccgcaccgc gatggccgcc | 1080 |
| cgccggatgc tgctggtgct ggacgacgcc cgcgacgcgg cccaggtctg gccgctgctg | 1140 |
| ccgaacaccg ccacctgccg ggtgctggtg acctcccgcg acccgctgcg cgagctggtc | 1200 |
| gcccgcagcg gggcggtgcc gctgcggctg ggcggcctcg ggttcgacga gtccgtggcg | 1260 |
| ctggtgcgcg gcatcatcgg cgaggcgcgg gccgggcgcg acccggacgc cctggtcggg | 1320 |
| ctggtcgagc tggtcgagct gtgcggtcgg gtgccgggcg cgctgctggc cgccgccgcg | 1380 |
| cacctggcca gcaaaccgca ctggggcgtg cccaggatgg tccgggagct caaccgcccg | 1440 |
| cgcagcaggc tgtccggcct cggcgggcag cacctgcgcg acgggctcgc ctccagcgcc | 1500 |
| cgctgcctgg acccggtggc ggccgacctg taccgggcgc tgggcggcct gcccacgccg | 1560 |
| gagctgacgt cctggacggc cacggccctg ctgggctgct cgacacccga ggccgacgac | 1620 |
| gtgctggagc gcctggtcga cgcgcacctg ctggagcccg ccggggcggg cgccggcggc | 1680 |
| gagagccact accggctgcc cagcctgtcc cacgcctacg cggcgaactt gccacgaccg | 1740 |
| gcccgtga | 1748 |

SEQ ID NO: 38

| | |
|---|---|
| cgcggatccc taagccgcaa tccctgattg | 30 |

SEQ ID NO: 39

| | |
|---|---|
| tccgatggac agtaaaagac tggcccccaa agcag | 35 |

SEQ ID NO: 40

| | |
|---|---|
| tgaggatcct tattacttgt cagctcgtcc atgccgagag tgatcc | 46 |

SEQ ID NO: 41

| | |
|---|---|
| cttttactgt ccatcggaac tagctatggt gagcaagggc gaggagctgt tcacc | 55 |

SEQ ID NO: 42

| | |
|---|---|
| tcaactgcta tcccccctgt ta | 22 |

SEQ ID NO: 43

| | |
|---|---|
| aaactccttt acttaaatgt tttgataaat aaa | 33 |

SEQ ID NO: 44

| | |
|---|---|
| tacatatggt gagcaagggc ga | 22 |

SEQ ID NO: 45

| | |
|---|---|
| tagaattctt atctagactt gtacagctcg | 30 |

SEQ ID NO: 46

| | |
|---|---|
| cggcgtttca cttctgagtt cggc | 24 |

SEQ ID NO: 47

| | |
|---|---|
| tagaattctt atctagactt gtacagctcg | 30 |

SEQ ID NO: 48

| | |
|---|---|
| tcaactgcta tcccccctgt tattaaaacg cttacattga ttattatagt catttaattt | 60 |
| taaatgtcta tacttttata aaataaatat aatcatattt ttttccggtt caccgtttta | 120 |
| taaatttttc tatggaagat tcattcataa tgtggtacac tcatcaacgg aaacgaatca | 180 |
| attaaatagc tattatcact tgtataacct caataatatg gtttgagggt gtctaccagg | 240 |
| aaccgtaaaa tcctgattac aaaatttgtt tatgacattt tttgtaatca ggattttttt | 300 |
| tatttatcaa aacatttaag taaaggagtt tgttatggtg agcaagggcg aggagctgtt | 360 |
| caccggggtg gtgcccatcc tggtcgagct ggacggcgac gtaaacggcc acaagttcag | 420 |
| cgtgtccggc gagggcgagg gcgatgccac ctacggcaag ctgaccctga agttcatctg | 480 |
| caccaccggc aagctgcccg tgccctggcc caccctcgtg accaccttcg gctacggcct | 540 |
| gcagtgcttc gcccgctacc ccgaccacat gaagcagcac gacttcttca agtccgccat | 600 |
| gcccgaaggc tacgtccagg agcgcaccat cttcttcaag gacgacggca actacaagac | 660 |
| ccgcgccgag gtgaagttcg agggcgacac cctggtgaac cgcatcgagc tgaagggcat | 720 |
| cgacttcaag gaggacggca acatcctggg gcacaagctg gagtacaact acaacagcca | 780 |
| caacgtctat atcatggccg acaagcagaa gaacggcatc aaggtgaact tcaagatccg | 840 |
| ccacaacatc gaggacggca gcgtgcagct cgccgaccac taccagcaga acacccccat | 900 |
| cggcgacggc cccgtgctgc tgcccgacaa ccactacctg agctaccagt ccgccctgag | 960 |
| caaagacccc aacgagaagc gcgatcacat ggtcctgctg gagttcgtga ccgccgccgg | 1020 |
| gatcactctc ggcatggacg agctgtacaa gtctagataa | 1060 |

SEQ ID NO: 49

| | |
|---|---|
| gtgagcaagg gcgaggagct gttcaccggg gtggtgccca tcctggtcga gctggacggc | 60 |
| gacgtaaacg gccacaagtt cagcgtgtcc ggcgagggcg agggcgatgc cacctacggc | 120 |
| aagctgaccc tgaagttcat ctgcaccacc ggcaagctgc ccgtgccctg gcccaccctc | 180 |
| gtgaccacct tcggctacgg cctgcagtgc ttcgcccgct accccgacca catgaagcag | 240 |
| cacgacttct tcaagtccgc catgcccgaa ggctacgtcc aggagcgcac catcttcttc | 300 |
| aaggacgacg gcaactacaa gacccgcgcc gaggtgaagt tcgagggcga caccctggtg | 360 |
| aaccgcatcg agctgaaggg catcaacttc aaggaggacg gcaacatcct ggggcacaag | 420 |
| ctggagtaca actacaacag ccacaacgtc tatatcatgg ccgacaagca gaagaacggc | 480 |
| atcaaggtga acttcaagat ccgccacaac atcgagggcg gcagcgtgca gctcgccgac | 540 |
| cactaccagc agaacacccc catcggcgac ggccccgtgc tgctgcccga caaccactac | 600 |
| ctgagctacc agtccgccct gagcaaagac cccaacgaga agcgcgatca catggtcctg | 660 |
| ctggagttcg tgaccgccgc cgggatcact ctcggcatgg acgagctgta caagtctaga | 720 |
| taa | 723 |

SEQ ID NO: 50

| | |
|---|---|
| gcgcggatcc tcacacctgg gggcgagctg | 30 |

SEQ ID NO: 51

| | |
|---|---|
| gcgccatatg atatctcctt cttaaagttc agcttgaatg aatctcttgc g | 51 |

SEQ ID NO: 52

| | |
|---|---|
| gcgccatatg gtgagcaagg gcgaggag | 28 |

SEQ ID NO: 53

| | |
|---|---|
| gcgcgtcgac ttatctagac ttgtacagct cgtc | 34 |

SEQ ID NO: 54

| | |
|---|---|
| cgatcctgac gcagattttt | 20 |

SEQ ID NO: 55

| | |
|---|---|
| ctcaccggct ccagatttat | 20 |

SEQ ID NO: 56

| | |
|---|---|
| ggatccttat tacttgtaca gctcgtccat gccgagagtg atcccggcgg cggtcacgaa | 60 |
| ctccagcagg accatgtgat cgcgcttctc gttggggtct ttgctcaggg cggactggta | 120 |
| gctcaggtag tggttgtcgg gcagcagcac ggggccgtcg ccgatggggg tgttctgctg | 180 |
| gtagtggtcg gcgagctgca cgctgccgcc ctcgatgttg tggcggatct tgaagttcac | 240 |
| cttgatgccg ttcttctgct tgtcggccat gatatagacg ttgtggctgt tgtagttgta | 300 |
| ctccagcttg tgccccagga tgttgccgtc ctccttgaag ttgatgccct tcagctcgat | 360 |
| gcggttcacc agggtgtcgc cctcgaactt cacctcggcg cgggtcttgt agttgccgtc | 420 |
| gtccttgaag aagatggtgc gctcctggac gtagccttcg ggcatggcgg acttgaagaa | 480 |
| gtcgtgctgc ttcatgtggt cggggtagcg ggcgaagcac tgcaggccgt agccgaaggt | 540 |
| ggtcacgagg gtgggccagg gcacgggcag cttgccggtg gtgcagatga acttcagggt | 600 |
| cagcttgccg taggtggcat cgccctcgcc ctcgccggac acgctgaact tgtggccgtt | 660 |
| tacgtcgccg tccagctcga ccaggatggg caccaccccg gtgaacagct cctcgccctt | 720 |
| gctcaccata tgatatctcc ttcttaaagt tcatctaggt ccgatggaca gtaaaagact | 780 |
| ggcccccaaa agcagacctg taatgaagat ttccatgatc accatcgtga cctatggaag | 840 |
| tacttaagta aaatgattgg ttcttaacat ggtttaatat agcttcatga accccattca | 900 |
| actggacact ttgctctcaa tcattgatga aggcagcttc gaaggcgcct ccttagccct | 960 |
| ttccatttcc ccctcggcgg tgagtcagcg cgttaaagct ctcgagcatc acgtgggtcg | 1020 |
| agtgttggta tcgcgcaccc aaccggccaa agcaaccgaa gcgggtgaag tccttgtgca | 1080 |
| agcagcgcgg aaaatggtgt tgctgcaagc agaaactaaa gcgcaactat ctggacgcct | 1140 |
| tgctgaaatc ccgttaacca tcgccatcaa cgcagattcg ctatccacat ggtttcctcc | 1200 |
| cgtgttcaac gaggtagctt cttggggtgg agcaacgctc acgctgcgct tggaagatga | 1260 |
| agcgcacaca ttatccttgc tgcggcgtgg agatgtttta ggagcggtaa cccgtgaagc | 1320 |
| taatcccgtg gcgggatgtg aagtagtaga acttggaacc atgcgccact tggccattgc | 1380 |
| aaccccctca ttgcgggatg cctacatggt tgatgggaaa ctagattggg ctgcgatgcc | 1440 |
| cgtcttacgc ttcggtccca aagatgtgct tcaagaccgt gacctggacg ggcgcgtcga | 1500 |
| tggtcctgtg gggcgcaggc gcgtatccat tgtcccgtcg gcggaaggtt ttggtgaggc | 1560 |
| aattcgccga ggccttggtt ggggacttct tcccgaaacc caagctgctc ccatgctaaa | 1620 |
| agcaggagaa gtgatcctcc tcgatgagat acccattgac acaccgatgt attggcaacg | 1680 |
| atggcgcctg gaatctagat ctctagctag actcacagac gccgtcgttg atgcagcaat | 1740 |
| cgagggattg cggccttagg tcgac | 1765 |

SEQ ID NO: 57

| | |
|---|---|
| ggatcccgag aaaggaaggg aagaaagcga aaggagcggg cgctagggcg ctggcaagtg | 60 |
| tagcggtcac gctgcgcgta accaccacac ccgccgcgct taatgcgccg ctacagggcg | 120 |
| cgtcccattc gccaatccgg atatagttcc tcctttcagc aaaaaacccc tcaagacccg | 180 |
| tttagaggcc ccaaggggtt atgctagtta ttgctcagcg gtggcagcag ccaactcagc | 240 |
| ttcctttcgg gctttgttag cagccggatc tcagtgggaa ttcctactgg aacaggtggt | 300 |
| ggcgggcctc ggcgcgctcg tactgctcca ccacggtgta gtcctcgttg tgggaggtga | 360 |
| tgtcgagctt gtagtccacg tagtggtagc cgggcagctt cacgggcttc ttggccatgt | 420 |
| agatggactt gaactcacac aggtagtggc cgccgccctt cagcttcagc gccatgtggt | 480 |
| tctcgccctt cagcacgccg tcgcgggggt agttgcgctc agtggagggc tcccagccca | 540 |
| gagtcttctt ctgcattacg gggccgtcgg aggggaagtt cacgccgatg aacttcacgt | 600 |
| ggtagatgag ggtgccgtcc tgcagggagg agtcctgggt cacggtcacc acgccgccgt | 660 |
| cctcgaagtt catcacgcgc tcccacttga agccctcggg gaaggactgc ttgaggtagt | 720 |
| cggggatgtc ggcggggtgc ttgatgtacg ccttggagcc gtagaagaac tggggggaca | 780 |
| ggatgtccca ggcgaagggc agggggccgc ccttggtcac ttgcagcttg gcggtctggg | 840 |
| tgccctcgta gggcttgccc tcgcccacgc cctcgatctc gaactcgtgg ccgttcacgg | 900 |
| agccctccat gtgcaccttg aagcgcatga agggcttgat gacgttctca gtgctatcca | 960 |
| tatgtatatc tccttctgca ggcatgcaag cttggcgtaa tcatggtcat atcttttaat | 1020 |
| tctgtttcct gtgtgaaatt gttatccgct cacaattcca cacattatac gagccgatga | 1080 |
| ttaattgtca acagctcatt tcagaatatt tgccagaacc gttatgatgt cggcgcaaaa | 1140 |
| aacattatcc agaacgggag tgcgccttga gcgacacgaa ttatgcagtg atttacgacc | 1200 |
| tgcacagcca taccacagct tccgatggct gcctgacgcc agaagcattg gtgcaccgtg | 1260 |
| cagtcgataa gcccggatca gcttgcaatt cgcgcgcgaa ggcgaagcgg catgcattta | 1320 |
| cgttgacacc atcgaatggt gcaaaacctt tcgcggtatg gcatgatagc gcccggaaga | 1380 |
| gagtcaattc agggtggtga atgtgaaacc agtaacgtta tacgatgtcg cagagtatgc | 1440 |
| cggtgtctct tatcagaccg tttcccgcgt ggtgaaccag gccagccacg tttctgcgaa | 1500 |
| aacgcgggaa aaagtggaag cggcgatggc ggagctgaat tacattccca accgcgtggc | 1560 |
| acaacaactg gcgggcaaac agtcgttgct gattggcgtt gccacctcca gtctggccct | 1620 |
| gcacgcgccg tcgcaaattg tcgcggcgat taaatctcgc gccgatcaac tgggtgccag | 1680 |
| cgtggtggtg tcgatggtag aacgaagcgg cgtcgaagcc tgtaaagcgg cggtgcacaa | 1740 |
| tcttctcgcg caacgcgtca gtgggctgat cattaactat ccgctggatg accaggatgc | 1800 |
| cattgctgtg gaagctgcct gcactaatgt tccggcgtta tttcttgatg tctctgacca | 1860 |
| gacacccatc aacagtatta ttttctccca tgaagacggt acgcgactgg gcgtggagca | 1920 |
| tctggtcgca ttgggtcacc agcaaatcgc gctgttagcg ggcccattaa gttctgtctc | 1980 |
| ggcgcgtctg cgtctggctg gctggcataa atatctcact cgcaatcaaa ttcagccgat | 2040 |
| agcggaacgg gaaggcgact ggagtgccat gtccggtttt caacaaacca tgcaaatgct | 2100 |
| gaatgagggc atcgttccca ctgcgatgct ggttgccaac gatcagatgg cgctgggcgc | 2160 |
| aatgcgcgcc attaccgagt ccgggctgcg cgttggtgcg gatatctcgg tagtgggata | 2220 |
| cgacgatacc gaagacagct catgttatat cccgccgtta accaccatca aacaggattt | 2280 |
| tcgcctgctg gggcaaacca gcgtggaccg cttgctgcaa ctctctcagg gccaggcggt | 2340 |
| gaagggcaat cagctgttgc ccgtctcact ggtgaaaaga aaaaccaccc tggcgcccaa | 2400 |
| tacgcaaacc gcctctcccc gcgcgtcggc cgccatgccg gcgataatgg cctgcttctc | 2460 |
| gccgaaacgt ttggtggcgg gaccagtgac gaaggcttga ggatcc | 2506 |

SEQ ID NO: 58

| | |
|---|---|
| gaacatcage gacaggacaa | 20 |

SEQ ID NO: 59

| | |
|---|---|
| gggaagcaaa gaaacgaaca | 20 |

SEQ ID NO: 60

| | |
|---|---|
| cctccccggg ttgatattag | 20 |

SEQ ID NO: 61

| | |
|---|---|
| ggccagcacg aatagcttta | 20 |

SEQ ID NO: 62

| | |
|---|---|
| aggaatctcc ctgcgtacaa | 20 |

SEQ ID NO: 63

| | |
|---|---|
| ceggattcat ccaagaaagc | 20 |

SEQ ID NO: 64

| | |
|---|---|
| gecttaaaac gccactcaat | 20 |

SEQ ID NO: 65

| | |
|---|---|
| ggccgttgat cattgttctt | 20 |

SEQ ID NO: 66

| | |
|---|---|
| aactccacgc tggagctcac | 20 |

SEQ ID NO: 67

| | |
|---|---|
| agaacgcgga gtccacg | 17 |

SEQ ID NO: 68

| | |
|---|---|
| MATTLLDLTK LIDGILKGSA QGVPAHAVGE QAIAAIGLDS SSLPTSDAIF AAVPGTRTHG | 60 |
| AQFAGTDNAA KAVAILTDAA GLEVLNEAGE TRPVIVVDDV RAVLGAASSS IYGDPSKDFT | 120 |
| FIGVTGTSGK TTTSYLLEKG LMEAGHKVGL IGTTGTRIDG EEVPTKLTTP EAPTLQALFA | 180 |
| RMRDHGVTHV VMEVSSHALS LGRVAGSHFD VAAFTNLSQD HLDFHPTMDD YFDAKALFFR | 240 |
| ADSPLVADKQ VVCVDDSWGQ RMASVAADVQ TVSTLGQEAD FSATDINVSD SGAQSFKINA | 300 |
| PSNQSYQVEL ALPGAFNVAN ATLAFAAAAR VGVDGEAFAR GMSKVAVPGR MERIDEGQDF | 360 |
| LAVVDYAHKP AAVAAVLDTL RTQTDGRLGV VTGAGGDRDS TKRGPMGQLS AQRADLVTVT | 420 |
| DDNPRSEVPA TIRAAVTAGA QQGASESERP VEVLEIGDRA EAIRVLVEWA QPGDGTVVAG | 480 |
| KGHEVGQLVA GVTHHFDDRE EVRAALTEKL NNKLPLTTEE G | 521 |

SEQ ID NO: 69

| | |
|---|---|
| atggcaacca cgttgctgga cctcaccaaa cttatcgatg gcatcctcaa gggctctgcc | 60 |
| cagggcgttc ccgctcacgc agtaggggaa caagcaatcg cggctattgg tcttgactcc | 120 |
| tccagcttac ctacctcgga cgctattttt gctgcagttc caggaacccg cactcacggc | 180 |
| gcacagtttg caggtacgga taacgctgcg aaagctgtgg ccattttgac tgacgcagct | 240 |
| ggacttgagg tgctcaacga agcaggagag acccgcccag tcatcgttgt tgatgatgtc | 300 |
| cgcgcagtac ttggcgcagc atcatcaagc atttatggcg atccttcaaa agatttcacg | 360 |
| ttcattggag tcactggaac ctcaggtaaa accaccacca gctacctctt ggaaaaagga | 420 |
| ctcatggagg caggccacaa agttggtttg atcggcacca caggtacacg tattgacggg | 480 |
| gaagaagtac ccacaaagct caccactcca gaagcgccga ctctgcaggc attgtttgct | 540 |
| cgaatgcgcg atcacggtgt cacccacgtg gtgatggaag tatccagcca tgcattgtca | 600 |
| ttgggcagag ttgcgggttc ccactttgat gtagctgcgt ttaccaacct gtcgcaggat | 660 |
| caccttgatt tccaccccac catggatgat tactttgacg cgaaggcatt gttcttccgc | 720 |
| gcagattctc cacttgtggc tgacaaacag gtcgtgtgcg tggatgattc ttggggtcag | 780 |
| cgcatggcca gcgtggcagc ggatgtgcaa acagtatcca cccttgggca agaagcagac | 840 |
| ttcagcgcta cagacatcaa tgtcagcgac tctggcgccc agagttttaa gatcaacgcc | 900 |
| ccctcaaacc agtcctacca ggtcgagcta gctcttccag gtgcgttcaa cgttgctaac | 960 |
| gccacgttgg catttgccgc tgcggcacgc gtgggtgttg atggcgaagc gtttgctcga | 1020 |
| ggcatgtcca aggtcgcggt tccaggccgt atggaacgca ttgatgaggg acaagacttc | 1080 |
| cttgcagtgg tggattatgc ccacaagcct gctgcagtgg ctgctgtgtt ggatacgttg | 1140 |
| aggacccaga ttgacgggcg cctcggagtg gttatcggtg ctggtggaga ccgcgattcc | 1200 |
| accaagcgtg gccccatggg gcagttgtcc gcacagcgtg ctgatctagt tattgtcact | 1260 |
| gatgacaacc ctcgttcaga ggtgcctgcc acgattcgcg cagcagtcac tgcaggagca | 1320 |
| cagcagggtg cttcagagtc cgaacgaccg gtggaagtcc tagaaattgg tgaccgtgca | 1380 |
| gaagcaattc gcgttttggt cgagtgggca cagcctggag atggcattgt agtagctgga | 1440 |
| aaaggccatg aagttggaca actagttgct ggtgtcaccc accattttga tgaccgcgaa | 1500 |
| gaagttcgcg ctgctttgac agaaaagctc aacaataaac ttccccttac tacggaagaa | 1560 |
| ggatag | 1566 |

SEQ ID NO: 70

| | |
|---|---|
| taggatcccg acaacatccc actgtctg | 28 |

SEQ ID N0: 71

| | |
|---|---|
| aagtcgacgt ctgcttcttg cccaagg | 27 |

SEQ ID NO: 72

| | |
|---|---|
| VSKGEELFTG VVPILVELDG DVNGHKFSVS GEGEGDATYG KLTLKFICTT GKLPVPWPTL | 60 |
| VTTFGYGLQC FARYPDHMKQ HDFFKSAMPE GYVQERTIFF KDDGNYKTRA EVKFEGDTLV | 120 |
| NRIELKGINF KEDGNILGHK LEYNYNSHNV YIMADKQKNG IKVNFKIRHN IEGGSVQLAD | 180 |
| HYQQNTPIGD GPVLLPDNHY LSYQSALSKD PNEKRDHMVL LEFVTAAGIT LGMDELYKSR | 240 |

What is claimed is:

1. A method for the identification of a cell having an increased intracellular concentration of a particular metabolite in a cell suspension, comprising the method steps:
i) provision of a cell suspension comprising cells which are genetically modified with respect to their wild type and which comprise a gene sequence coding for an autofluorescent protein, wherein the expression of the autofluorescent protein depends on the intracellular concentration of a particular metabolite;
ii) genetic modification of the cells to obtain a cell suspension in which the cells differ with respect to the intracellular concentration of a particular metabolite;
iii) identification of individual cells in the cell suspension having an increased intracellular concentration of this particular metabolite by detection of the intracellular fluorescence activity.

2. The method according to claim 1, wherein the genetic modification in method step ii) is carried out by non-targeted mutagenesis.

3. The method according to claim 1, further comprising the method step:
iv) separating off of the identified cells from the cell suspension.

4. The method according to claim 3, wherein the separating off is carried out by means of flow cytometry.

5. The method according to claim 1, wherein control of the expression of the gene sequence coding for the autofluorescent protein is effected as a function of the intracellular concentration of the particular metabolite at the transcription level.

6. The method according to claim 1, wherein the gene sequence coding for the autofluorescent protein is under the control of a heterologous promoter which, in the wild type of the cell, controls the expression of a gene of which the expression in the wild-type cell depends on the intracellular concentration of a particular metabolite.

7. The method according to claim 6, wherein control of the expression of the gene sequence coding for the autofluorescent protein is effected as a function of the intracellular concentration of the particular metabolite at the translation level.

8. The method according to claim 5, wherein the gene sequence coding for the autofluorescent protein is bonded functionally to a DNA sequence which, at the mRNA level, assumes the function of a riboswitch which regulates the expression of the gene sequence coding for the autofluorescent protein at the transcription level or the translation level.

9. The method according to claim 1, wherein the cell is a cell of the genus Corynebacterium or Escherichia.

10. The method according to claim 1, wherein the metabolite is chosen from the group consisting of amino acids, nucleotides, fatty acids and carbohydrates.

11. The method according to claim 10, wherein the metabolite is an amino acid.

12. The method according to claim 11, wherein the amino acid is L-lysine.

13. The method according to claim 5, wherein the promoter is the lysE promoter and the gene is the lysE gene.

14. The method according to claim 1, wherein the autofluorescent protein is green fluorescent protein (GFP) or a variant of this protein.

15. A method for the production of a cell which is genetically modified with respect to its wild type with optimized production of a particular metabolite, comprising the method steps:
I) provision of a cell suspension comprising cells which are genetically modified with respect to their wild type and which comprise a gene sequence coding for an autofluorescent protein, wherein the expression of the autofluorescent protein depends on the intracellular concentration of a particular metabolite;
II) genetic modification of the cells to obtain a cell suspension in which the cells differ with respect to their intracellular concentration of a particular metabolite;
III) identification of individual cells in the cell suspension having an increased intracellular concentration of the particular metabolite by detection of the intracellular fluorescence activity;
IV) separating off of the identified cells from the cell suspension;
V) identification of those genetically modified genes G₁ to Gₙ or those mutations M₁ to Mₘ in the cells identified and separated off which are responsible for the increased intracellular concentration of the particular metabolite;
VI) production of a cell which is genetically modified with respect to its wild type with optimized production of the particular metabolite, of which the genome comprises at least one of the genes G₁ to Gₙ and/or at least one of the mutations M₁ to Mₘ.

16. The method according to claim 15, wherein the genetic modification in method step II) is carried out by non-targeted mutagenesis.

17. The method according to claim 15, wherein control of the expression of the gene sequence coding for the autofluorescent protein is effected as a function of the intracellular concentration of the particular metabolite at the transcription level.

18. The method according to claim 15, wherein the gene sequence coding for the autofluorescent protein is under the control of a heterologous promoter which, in the wild type of the cell, controls the expression of a gene of which the expression in the wild-type cell depends on the intracellular concentration of a particular metabolite.

19. The method according to claim 18, wherein control of the expression of the gene sequence coding for the autofluorescent protein is effected as a function of the intracellular concentration of the particular metabolite at the translation level.

20. The method according to claim 17, wherein the gene sequence coding for the autofluorescent protein is bonded functionally to a DNA sequence which, at the mRNA level, assumes the function of a riboswitch which regulates the expression of the gene sequence coding for the autofluorescent protein at the transcription level or the translation level.

21. The method according to claim 15, wherein the cell is a cell of the genus Corynebacterium or Escherichia.

22. The method according to claim 15, wherein the metabolite is chosen from the group consisting of amino acids, nucleotides, fatty acids and carbohydrates.

23. The method according to claim 22, wherein the metabolite is an amino acid.

24. The method according to claim 23, wherein the amino acid is L-lysine.

25. The method according to claim 17, wherein the promoter is the lysE promoter and the gene is the lysE gene.

26. The method according to claim 15, wherein the autofluorescent protein is green fluorescent protein (GFP) or a variant of this protein.
